(19) United States
(12) Patent Application Publication
(10) Pub. No.: US 2022/0000974 A1
(43) Pub. Date: Jan. 6, 2022

(54) Targeting CD24-Siglec Interactions for Treating Subjects with Prediabetes or Diabetes

(71) Applicants: Oncolmmune, Inc., Rockville, MD (US); Children's Research Institute, Children's National Medical Center, Washington, DC (US)

(72) Inventors: Yang Liu, Washington, DC (US); Pan Zheng, Washington, DC (US); Xu Wang, Washington, DC (US); Martin Devenport, Gaithersburg, MD (US)

(21) Appl. No.: 17/428,784

(22) PCT Filed: Feb. 5, 2020
(86) PCT No.: PCT/US2020/016874

§ 371 Date: Aug. 5, 2021

Related U.S. Application Data
(60) Provisional application No. 62/801,972, filed on Feb. 6, 2019.

(51) Int. Cl. A61K 38/17; C07K 14/705; A61P 3/06; A61P 3/10

(52) U.S. Cl. CPC A61K 38/177; C07K 14/70596; C07K 2319/30; A61P 3/10; A61P 3/06

(57) ABSTRACT

Provided herein are methods and compositions for lowering low-density lipoprotein cholesterol or glucose levels, and for treating subjects with prediabetes or diabetes by targeting CD24-Siglec interactions.

### STATEMENT REGARDING FEDERALLY SPONSORED RESEARCH OR DEVELOPMENT

This invention was made in part with Government support under SBIR Grant Number 1R44CA221513 awarded by the National Cancer Institute. The Government has certain rights in this invention.

### FIELD OF THE INVENTION

The present invention relates to the use of a CD24 protein or targeting CD24-Siglec interactions for treating subjects with prediabetes or diabetes.

### BACKGROUND OF THE INVENTION

Diabetes, and the risk of developing diabetes, remains an enormous health issue. Prediabetes is the condition in which the blood sugar levels are higher than normal but not yet high enough to be type 2 diabetes. Without lifestyle changes, people with prediabetes are very likely to progress to type 2 diabetes and the long-term damage of diabetes, especially to the heart, blood vessels and kidneys, may already be starting. According to the CDC National Diabetes Statistics Report (2017), an estimated 33.9% of U.S. adults aged 18 years or older (84.1 million people) had prediabetes in 2015, based on their fasting glucose or HbA1C level. Nearly half (48.3%) of adults aged 65 years or older had prediabetes. Among adults with prediabetes, 11.6% reported being told by a health professional that they had this condition. Age-adjusted data for 2011-2014 indicated that more men (36.6%) than women (29.3%) had prediabetes. Prevalence of prediabetes was similar among racial and ethnic groups. An estimated 30.3 million people of all ages—or 9.4% of the U.S. population—had diabetes in 2015. This total included 30.2 million adults aged 18 years or older (12.2% of all U.S. adults), of which 7.2 million (23.8%) were not aware of or did not report having diabetes. The percentage of adults with diabetes increased with age, reaching a high of 25.2% among those aged 65 years or older. Compared to non-Hispanic whites, the age-adjusted prevalence of diagnosed and undiagnosed diabetes was higher among Asians, non-Hispanic blacks, and Hispanics during 2011-2014.

People with diabetes are more prone to having unhealthy high cholesterol levels, a common condition called diabetic dyslipidemia. This condition contributes to cardiovascular disease (CVD) and stroke. Reducing the burden of CVD in diabetes is a major challenge for physicians. Major advances have been made to control lipid and glucose metabolism. However, the disorders in lipid and glucose metabolism are treated by drugs specific for either lipid or glucose metabolism. Elevated LDL-C is most frequently treated with statins (3-hydroxy-3-methylglutaryl-co-enzyme-A reductase inhibitors). However, long-term follow up studies have demonstrated that statin inhibitors increase diabetes risk. In support of this notion, recent studies have revealed that genetic variants of the statin target, HMGCR, showed the opposite trend of LDL-C levels and increased diabetes risk. Although it is unclear whether PCSK9 inhibitors also increase diabetes risk, genetic data demonstrates that patients with hypomorphic variants of PCSK9 have an increased risk of diabetes. For these reasons, physicians have strong reservations about prescribing available LDL-C-lowering drugs to prediabetic patients. Thus, there is a large unmet medical need for drugs that lower LDL-C levels in prediabetic patients while either reducing, or at least not increasing, the risk of diabetes. Drugs that can simultaneously treat disorders associated with both lipid and glucose metabolisms would be particularly useful.

One of the hallmarks of metabolic diseases, such as prediabetes and diabetes, is a chronic low-grade systemic inflammation. During the process of obesity, there is an increased accumulation of inflammatory cells in metabolic tissues, particularly in liver and adipose tissue. This chronic tissue inflammation causes increased levels of proinflammatory cytokines that impair insulin signaling and disrupt systemic metabolic homeostasis. Many studies have indicated that metabolic regulation and immune response pathways are highly integrated. A number of genes, previously thought to work specifically in the immune system, are now considered crucial regulators of metabolism. To date, the master inflammatory signaling pathways such as NF-κB and JNK pathways have been found to regulate insulin sensitivity and metabolic homeostasis. Targeted deletion of the kinases, IKKβ or IKKε, which negatively regulate the inhibitory IκB proteins, can improve insulin sensitivity in obese mice. For the innate immune system, pattern recognition receptors, which recognize both pathogen- and damage-associated molecular patterns (PAMPs and DAMPs, respectively), are also involved in metabolic regulation, such as the Toll-like receptors (TLRs) and NOD-like receptors (NLRs), which indicates a close relationship between nutrient and pathogen response systems. For this reason, there has been a surge of interest in targeting inflammation to treat metabolic disorders and its associated CVD.

### SUMMARY OF THE INVENTION

Provided herein is a method of treating a prediabetic or diabetic subject. The treatment may be for reducing serum LDL-C or glucose levels, or reducing the risk of CVD, diabetes or atherosclerotic cardiovascular disease events. The method may comprise administering a CD24 protein or a Siglec agonist to a subject in need thereof. The inventors have discovered sialoside-based pattern recognition as a negative regulator for inflammation caused by DAMPs. Specifically, they have found that through its sialic acid, CD24 interacts with Siglec G in mice and Siglec 10 in human to suppress host response to tissue injury. Now they have demonstrated that the CD24 interaction with Siglec E in mice, but not with other Siglecs tested, controls metabolic homeostasis of both glucose and lipids.

Clinical and pre-clinical data demonstrate that a single dose of CD24Fc has the ability to simultaneously lower serum LDL-C and glucose, while also increasing leptin levels. This indicates that CD24Fc could be used to manage the high risk for cardiovascular disease (CVD) and stroke, as well as diabetes, in prediabetic subjects. This is a differentiation from current standard of care in which statins are typically used to lower LDL-C levels (but with the increased risk of CVD) and different drugs, such as empagliflozin (JARDIANCE®) are used to manage serum glucose levels. In addition, the inventors have discovered an unexpected and surprising function of agonizing Siglecs using CD24Fc in normalizing both lipid and glucose metabolism.

Targeted deletion of the Cd24 gene in mice exacerbated metabolic syndromes, including increased weight gain due to fat content, impaired glucose and lipid homeostasis, as indicated by remarkably increased fasting blood glucose, LDL-C and TC levels. In addition, of all Siglec mutations studied, only mutation of Siglece fully recapitulates the metabolic phenotype of the CD24 mutation. The essentially identical phenotypes, and the physical interaction between CD24 and Siglec E, indicate that Siglec E is the functional CD24 receptor in mice and, consistent with this notion, CD24Fc suppresses metabolic syndrome in a Siglece-dependent mechanism.

Taken together, the ability to simultaneously lower serum LDL-C and glucose, while also increasing leptin levels, indicates that CD24Fc could be used to manage the high risk for CVD and stroke in prediabetic and diabetic subjects.

Provided herein is a method of treating a subject in need thereof with diabetes, prediabetes or at risk of developing diabetes. The method may comprise administering a CD24 protein or Siglec agonist to the subject. The method may be for reducing serum LDL-C or blood glucose levels, or both, and restoring metabolic homeostasis. The method may also be for treating CVD, or reducing the risk of CVD, diabetes or an atherosclerotic CVD event.

The subject may have impaired fasting glucose or impaired glucose tolerance. The subject may have at least one of a hemoglobin A1C level of 5.7-6.4%, a fasting plasma glucose level of 100-125 mg/dL, and a glucose level of 140-199 mg/dL in a 2-hour post 75 g oral glucose challenge. The subject may have diabetes. The subject may have at least one of a fasting plasma glucose level of ≥126 mg/dL, a hemoglobin A1C level of ≥6.5%, and a glucose level of ≥200 mg/dL in a 2-hour 75 g oral glucose challenge. The subject may have insulin insensitivity.

The subject may have an elevated LDL-C level. The subject may have a LDL-C level greater than or equal to 70 mg/dL, 75 mg/dL, or 190 mg/dL. The subject may have been previously treated with another LDL-C-lowering drug, wherein the other LDL-C-lowering drug is not a CD24 protein. The other LDL-C-lowering drug may be a statin or an antagonist of PCSK9.

The CD24 protein may comprise a mature human CD24 polypeptide or a variant thereof. The mature human CD24 polypeptide may comprise the sequence set forth in SEQ ID NO: 1 or 2. The CD24 protein may comprise a protein tag. The protein tag may be fused at the N- or C-terminus of the CD24 protein. The protein tag may comprise a portion of a mammalian immunoglobulin (Ig) protein. The portion of the Ig protein may be a Fc region of a human Ig protein. The Fc region may comprise a hinge region and CH2 and CH3 domains of IgG1, IgG2, IgG3, IgG4, or IgA. The Fc region may comprise a hinge region and CH2, CH3 and CH4 domains of IgM. The CD24 protein may comprise the sequence set forth in SEQ ID NO: 6, 11 or 12. The amino acid sequence of the CD24 protein may consist of the sequence set forth in SEQ ID NO: 6, 11 or 12. The CD24 protein may be soluble, and may be glycosylated.

The Siglec agonist may be characterized by its ability to induce association of tyrosine phosphorylation in one or more Immunoreceptor tyrosine-based inhibitor motif (ITIM) domains of a Siglec. The Siglec may be Siglec E or a functional homolog thereof. The functional homolog may be human, and may be one or more of Siglec 6-9 and 12.

### BRIEF DESCRIPTION OF THE DRAWINGS

FIG. 1A shows the amino acid composition of the full length CD24 fusion protein, CD24Fc (also referred to herein as CD24Ig) (SEQ ID NO: 5). The underlined 26 amino acids are the signal peptide of CD24 (SEQ ID NO: 4), which are cleaved off during secretion from a cell expressing the protein and thus missing from the processed version of the protein (SEQ ID NO: 6). The bold portion of the sequence is the extracellular domain of the mature CD24 protein used in the fusion protein (SEQ ID NO: 2). The last amino acid (A or V) that is ordinarily present in the mature CD24 protein has been deleted from the construct to avoid immunogenicity. The non-underlined, non-bold letters are the sequence of IgG1 Fc, including the hinge region and CH1 and CH2 domains (SEQ ID NO: 7). FIG. 1B shows the sequence of CD24^{V}Fc (SEQ ID NO: 8), in which the mature human CD24 protein (bold) is the valine polymorphic variant of SEQ ID NO: 1. FIG. 1C shows the sequence of CD24^{A}Fc (SEQ ID NO: 9), in which the mature human CD24 protein (bold) is the alanine polymorphic variant of SEQ ID NO: 1. The various parts of the fusion protein in FIGS. 1B and 1C are marked as in FIG. 1A and the variant valine/alanine amino acid is double underlined.

FIG. 2 shows amino acid sequence variations between mature CD24 proteins from mouse (SEQ ID NO: 3) and human (SEQ ID NO: 2). The potential O-glycosylation sites are bolded, and the N-glycosylation sites are underlined.

FIGS. 3A-C show WinNonlin compartmental modeling analysis of pharmacokinetics of CD24Fc (CD24Ig) in mice. The opened circles represent the average of 3 mice, and the line is the predicted pharmacokinetic curve. FIG. 3A. i.v. injection of 1 mg CD24Fc. FIG. 3B. s.c. injection of 1 mg CD24Fc. FIG. 3C. Comparison of the total amounts of antibody in the blood as measured by areas under curve (AUC), half-life and maximal blood concentration. Note that overall, the AUC and Cₘₐₓ of the s.c. injection is about 80% of i.v. injection, although the difference is not statistically significant.

FIG. 4 shows CD24Fc Decreased Human Serum LDL-C in Healthy Subjects. Serum samples were taken from Subjects in the Phase I trial involving subjects receiving single CD24Fc doses: 0, 10, 30, 60, 120, 240 mg. The level of LDL-C measured at pre-dosing baseline, and 7, 14 or 42 days after dosing. Based on linear regression analysis, dose-dependent reduction of LDL-C was observed on days 7 and 14 among patients receiving 30-240 mg of CD24Fc (***P<0.0001). When compared with placebo control, 240 mg of CD24Fc significantly reduced LDL-C at days 7 and 14 (*, P<0.05).

FIG. 5 shows the ratio of leptin on day3/day-1 for patients grouped by dosing cohort. Serum samples (pre-dosing and day 3 after dosing) were taken from Subjects in the Phase I trial involving subjects receiving single CD24Fc doses: 0, 10, 30, 60, 120, 240 mg. The levels of leptin were measured at pre-dosing baseline, and 3 days after dosing. Based on linear regression analysis, dose-dependent induction of leptin was observed in patients receiving placebo, 60, 120 and 240 mg of CD24Fc (P=0.009). When compared with placebo control, 240 mg of CD24Fc significantly induced leptin on day 3 (P=0.05).

FIG. 6 shows a plot of mean plasma CD24Fc concentration (±SD) by treatment for a PK Evaluable Population in human subjects. PK=pharmacokinetic; SD=standard deviation.

FIG. 7 shows a dose proportionality plot of CD24Fc Cₘₐₓ versus dose for a PK Evaluable Population.

FIG. 8 shows a dose proportionality plot of CD24Fc AUC_{0-42d} versus dose for a PK Evaluable Population.

FIG. 9 shows a dose proportionality plot of CD24Fc AUC_{0-inf} versus dose for a PK Evaluable Population.

FIG. 10 shows single dosing of CD24Fc reduces LDL-C levels in hematopoietic stem cell transplantation (HCT) patients. The study involved three arms: placebo control (N=6), 240 mg single dosing (N=6) and 480 mg single dosing (N=12, as the samples from multi-dosing cohort patients after receiving the first dosing are included). Data shown are % of pre-dosing LDL-C levels at 14 days after HCT (15 days after CD24Fc or placebo dosing). Statistical significance (P values) was calculated by two tailed t-tests.

FIGS. 11A-H show that CD24Fc improves glucose and lipid homeostasis in mice. WT mice were fed with HFD for 3 weeks, then treated with CD24Fc or control IgG for 2 weeks. (A-E) Blood glucose, total cholesterol, LDL-C, HDL-C and triglyceride levels in 6 hr fasted mice. (F-H) TC/HDL-C, LDL-C/HDL-C and TG/HDL-C were calculated. n=7 per group. All values are expressed as mean±SD. *P<0.05, **P<0.01, ***P<0.001 by unpaired Student's t-test.

FIGS. 12A-C show that CD24 binds to all but one ITIM-containing Siglec and provides a physiological stimulus for Siglecs G and E. a. Interaction between endogenously expressed CD24 with recombinant Siglecs. Spleen cell lysate was incubated in 96-well plates pre-coated with indicated Siglec-Fc or control IgG Fc (1 μg/well). After washing away unbound molecules, the amount of CD24 was detected with biotinylated anti-CD24 mAb (M1/69) followed by HRP-conjugated streptavidin. Data shown are means and SD of OD450 and are based on repeating experiments twice. The letter in the bar indicates cellular expression of Siglecs: B, B cells; N, neutrophils; cDC, conventional dendritic cells; pDC, plasmocytoid DC; M, monocytes; Eo, eosinophils; Mac, macrophages. b. Direct interaction between CD24 and Siglecs. As in (a), except biotinylated CD24Fc was added instead of cell lysate. c. Targeted mutation of CD24 abrogates the endogenous association between Siglec G and E with SHP-1. WT and CD24^{-/-} spleen cell lysate was immunoprecipitated with control IgG, anti-Siglec G, or anti-Siglec E. The amount of co-precipitated Siglec and SHP-1 was determined by Western blot. The total amount of all proteins in the spleen cell lysate was detected by Western blot (right panels).

FIGS. 13A-H show the identification of negative regulators of metabolic disorder among potential CD24 receptors. Fasting blood glucose (A) and total cholesterol levels (B) of mice with single or combined mutations of the given Siglec genes. (C-J). Metabolic disorder of Siglec-E KO mice when they were fed with normal diet. (C) Body weight over 12 month period and a photograph of representative mice at 8 months. (D) Body composition was detected by dual energy X-ray absorptiometry (DEXA). (E-H) total cholesterol (E), triglycerides (F), blood glucose (G) in mouse plasma after 6 hours of fasting. (H) Glucose tolerance of WT and Siglec-E KO mice. The corresponding area under the curve (AUC) of the blood glucose levels in each group was calculated. All values are expressed as mean±SEM. *P<0.05, **P<0.01, ***P<0.001, unpaired Student's t-test. Data shown are representative of two or three independent experiments.

FIG. 14 shows a single injection of CD24Fc (100 μg/mouse) reduces fasting glucose levels within 3 days. WT and Siglece^{-/-} mice were treated with either control IgG or CD24Fc i.p. Six-hour fasting glucose levels were measured on day 3 after the CD24Fc treatment.

FIGS. 15A-C show CD24Fc alters glucose and lipid levels in mouse plasma. A. Diagram of the experimental design. WT and Siglece^{-/-} male mice were fed with HFD starting at the age of 8 weeks old for 4 weeks. Mice were then injected intraperitoneally with CD24Fc (100 μg per dose) or an equivalent amount of isotype control IgG twice a week for 2 weeks. Fasting blood glucose and lipid contents were detected before (day 0) and after (day 14) of CD24Fc or IgG treatments. B. Glucose and lipid levels on day 14. C. ratios of lipid and glucose levels (day 14 over day 0).

FIGS. 16A-B show that Siglec E is necessary for suppression of inflammatory cytokine gene expression by macrophages. Peritoneal macrophages from WT or Siglece^{-/-} mice were stimulated with 500 μM of paltimic fatty acids in the presence of control IgG or CD24Fc (10 μg/ml) for 16 hours, and the mRNA for TNF-α (A) and IL-6 (B) were measured by RT-qPCR.

FIGS. 17A-D show that CD24Fc therapy improves metabolic disorders in DIO mice. Male C57/BL6/NCr mice were fed with HFD for 8 weeks, then treated with CD24Fc (100 μg per dose) or control IgGFc twice a week for 4 more weeks. n=7 per group. FIG. 17A. Body weight. FIGS. 17B-C. Blood glucose, TC, TG, LDL-C, HDL-C and FFA levels were detected after 6 hr of fasting. FIG. 17D. GTT and ITT data for mice.

### DETAILED DESCRIPTION

The inventors have found that, surprisingly, CD24-Siglec interactions control metabolic homeostasis of both glucose and lipid. Accordingly, proteins containing a mature CD24 sequence are effective for simultaneously lowering serum LDL-C and glucose levels, and are additionally useful for treating and/or preventing atherosclerosis, and for reducing the risk of cardiovascular disease such as atherosclerotic cardiovascular disease.

Initial safety of CD24Fc in healthy human subjects was initially demonstrated through a Phase I, randomized, double-blind, placebo-controlled, single ascending dose study that was conducted to assess the safety, tolerability, and PK of CD24Fc in healthy male and female adult subjects (ClinicalTrials.gov Identifier: NCT02650895). This study showed that the single dose of IV administration of CD24Fc up to 240 mg was safe and well tolerated in healthy subjects. CD24Fc has also been tested in a Phase II clinical study for the prophylaxis of acute GvHD in cancer patients undergoing allogeneic myeloablative hematopoietic stem cell transplantation (HCT). The Phase IIa portion of the trial (ClinicalTrials.gov Identifier: NCT02663622) was a randomized double blind trial comprising three single ascending dose cohorts (240 mg and 480 mg) and a single multi-dose cohort (480 mg (day −1), 240 mg (day +14) and 240 mg (day +28)) of CD24Fc in addition to SOC GVHD prophylaxis. The Phase II study has shown that IV administration of CD24Fc up to 480 mg as a single dose and in a multi-dose regimen is generally well tolerated in the intent-to-treat (ITT) population.

Using serum samples from the Phase I study in healthy subjects, a number of analytes were assayed to determine changes from baseline following CD24Fc administration. In particular, statistically-significant dose-dependent changes in serum LDL-C and leptin were observed after a single dosing of CD24Fc. Based on the data observed in the Phase I study in healthy subjects, the effect of CD24Fc on serum LDL-C and leptin, as well as other analytes related to fat metabolism continued to be studied in the Phase II GvHD prophylaxis study. Statistically significant decreases in LDL-C levels in patients receiving a single dose of 240 mg or 480 mg CD24Fc, as compared with a placebo control, have again been observed.

This observation was recapitulated and expanded in multiple mouse models and, with the benefit of less variation in animal models, a broad impact of CD24Fc in treating multiple abnormalities was observed. High-fat diet fed mice were treated with CD24Fc and the effect of CD24Fc was measured on total cholesterol, LDL-C and fasting glucose levels. The data demonstrate that CD24Fc reduced total cholesterol, LDL-C and fasting glucose. These results were confirmed using a CD24 knockout mouse model in which the CD24 gene knockout increased cholesterol levels, and treatment with CD24Fc reduced LDL-C levels. Furthermore, the knockout mice demonstrated an increase in overall body weight and corresponding increase in % body fat relative to wild type mice. In addition, multiple mouse strains were generated with single or combined mutations of Siglech and the CD33-family Siglec genes to show that the CD24-Siglec E interaction, but not those with other Siglecs tested, controls metabolic homeostasis of both glucose and lipid. Siglec-E knockout mice displayed higher fasting blood glucose and total cholesterol levels compared to wild-type mice, resulting in increased weight gain and fat content, as well as exhibiting defects in the glucose tolerance test. Thus, targeted mutations of the CD24 and Siglece genes caused, while CD24Fc treatment reduced in Siglece-dependent manner, metabolic disorders in mice, including hyperlipidemia, hyperglycosemia and insulin resistance as well as non-alcoholic steatosis hepatitis. This data not only revealed a missing link between inflammation and metabolic syndromes, but also provide a therapeutic approach to simultaneously correct disorders in glucose and lipid metabolisms.

Therefore, CD24Fc treatment results in a reprogramming of lipid metabolism. Also, unlike other pharmaceutical interventions of dyslipidemia which either has been proven to or has the potential to cause hyperglycemia, CD24Fc treatment also reduced fasting blood glucose, improved glucose tolerance and reduce insulin resistance. This is a most valuable feature of CD24Fc that differentiates it from current therapeutics, including Statins and PSCK9 inhibitors, whose utility may be restrained for prediabetes for fear of causing diabetes.

CD24Fc-based metabolic reprogramming differs from the dominant approach that specifically target systemic LDL-C levels by targeting either synthesis or uptake of LDL-C. By lowering inflammatory responses in the liver and adipose tissues, CD24Fc may help to clear out a major root cause of metabolic syndrome. It should be noted, however, that other biological drugs that target specific inflammatory cytokines, while effective for either hyperlipidemia or hyperglycemia have failed to simultaneously regulate both.

People at risk for type 2 diabetes often have impaired glucose tolerance (IGT), a pre-diabetic state of hyperglycemia that is associated with insulin resistance and increased risk of cardiovascular pathology. Furthermore, several statins have been shown to increase insulin resistance indices, glucose levels and glycosylated hemoglobin (HbHbA1c). The treatment of increased glucose levels may require additional therapies, often in combination with statins such as empagliflozin (JARDIANCE®), which can have its own side effects and risks. This suggests that the ability to control LDL-C and glucose levels through a new pathway may have potential advantages over existing therapies.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

A “peptide” or “polypeptide” is a linked sequence of amino acids and may be natural, synthetic, or a modification or combination of natural and synthetic.

“Substantially identical” may mean that a first and second amino acid sequence are at least 60%, 65%, 70%, 75%, 80%, 85%, 90%, 95%, 96%, 97%, 98%,or 99% over a region of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids.

“Treatment” or “treating,” when referring to protection of an animal from a disease, means preventing, suppressing, repressing, or completely eliminating the disease. Preventing the disease involves administering a composition of the present invention to an animal prior to onset of the disease. Suppressing the disease involves administering a composition of the present invention to an animal after induction of the disease but before its clinical appearance. Repressing the disease involves administering a composition of the present invention to an animal after clinical appearance of the disease.

A “variant” may mean a peptide or polypeptide that differs in amino acid sequence by the insertion, deletion, or conservative substitution of amino acids, but retain at least one biological activity. Representative examples of “biological activity” include the ability to bind to a toll-like receptor and to be bound by a specific antibody. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

### 2. CD24

Provided herein is a CD24 protein, which may comprise the amino acid sequence of mature human CD24 or those from other mammals, which corresponds to the extracellular domain (ECD) of CD24, or a variant thereof. As described above, the sequence of the mature human CD24 protein is 31 amino acids long with a variable alanine (A) with valine (V) residue at its C-terminal end:

(SEQ ID NO: 1)
SETTTGTSSNSSQSTSNSGLAPNPTNATTK(V/A)

The C-terminal valine or alanine may be immunogenic and may be omitted from the CD24 protein to reduce its immunogenicity. Therefore, the CD24 protein may comprise the amino acid sequence or mature human CD24 lacking the C-terminal amino acid:

(SEQ ID NO: 2)
SETTTGTSSNSSQSTSNSGLAPNPTNATTK

Despite considerable sequence variations in the amino acid sequence of the mature CD24 proteins from mouse and human, they are functionally equivalent, as human CD24Fc has been shown to be active in the mouse. The amino acid sequence of the human CD24 ECD shows some sequence conservation with the mouse protein (39% identity; Genbank accession number NP_033976). However, it is not that surprising that the percent identity is not higher as the CD24 ECD is only 27-31 amino acids in length, depending on the species, and binding to some of its receptor(s), such as Siglec 10/G, is mediated by its sialic acid and/or galactose sugars of the glycoprotein. The amino acid sequence identity between the extracellular domains of the human Siglec-10 (GenBank accession number AF310233) and its murine homolog Siglec-G (GenBank accession number NP_766488) receptor proteins is 63%. As a result of sequence conservation between mouse and human CD24 primarily in the C-terminus and in the abundance of glycosylation sites, significant variations in the mature CD24 proteins may be tolerated in using the CD24 protein, especially if those variations do not affect the conserved residues in the C-terminus or do not affect the glycosylation sites from either mouse or human CD24. Therefore, the CD24 protein may comprise the amino acid sequence of mature murine CD24:

(SEQ ID NO: 3)
NQTSVAPFPGNQNISASPNPTNATTRG.

The amino acid sequence of the human CD24 ECD shows more sequence conservation with the cynomolgus monkey protein (52% identity; UniProt accession number UniProtKB-I7GKK1) than with mouse. Again, this is not surprising given that the percent identity is not higher as the ECD is only 29-31 amino acids in length in these species, and the role of sugar residues in binding to its receptor(s). The amino acid sequence of cynomolgous Siglec-10 receptor has not been determined but the amino acid sequence identity between the human and rhesus monkey Siglec-10 (GenBank accession number XP_001116352) proteins is 89%. Therefore, the CD24 protein may also comprise the amino acid sequence of mature cynomolgous (or rhesus) monkey CD24:

(SEQ ID NO: 10)
TVTTSAPLSSNSPQNTSTTPNPANTTTKA

The CD24 protein may be soluble. The CD24 protein may further comprise an N-terminal signal peptide, to allow secretion from a cell expressing the protein. The signal peptide sequence may comprise the amino acid sequence MGRAMVARLGLGLLLLALLLPTQIYS (SEQ ID NO: 4). Alternatively, the signal sequence may be any of those that are found on other transmembrane or secreted proteins, or those modified from the existing signal peptides known in the art.

### a. Fusion

The CD24 protein may be fused at its N- or C-terminal end to a protein tag, which may comprise a portion of a mammalian Ig protein, which may be human or mouse or another species. The portion may comprise an Fc region of the Ig protein. The Fc region may comprise at least one of the hinge region, CH2, CH3, and CH4 domains of the Ig protein. The Ig protein may be human IgG1, IgG2, IgG3, IgG4, or IgA, and the Fc region may comprise the hinge region, and CH2 and CH3 domains of the Ig. The Fc region may comprise the human immunoglobulin G1 (IgG1) isotype SEQ ID NO: 7. The Ig protein may also be IgM, and the Fc region may comprise the hinge region and CH2, CH3, and CH4 domains of IgM. The protein tag may be an affinity tag that aids in the purification of the protein, and/or a solubility-enhancing tag that enhances the solubility and recovery of functional proteins. The protein tag may also increase the valency of the CD24 protein. The protein tag may also comprise GST, His, FLAG, Myc, MBP, NusA, thioredoxin (TRX), small ubiquitin-like modifier (SUMO), ubiquitin (Ub), albumin, or a Camelid Ig. Methods for making fusion proteins and purifying fusion proteins are well known in the art.

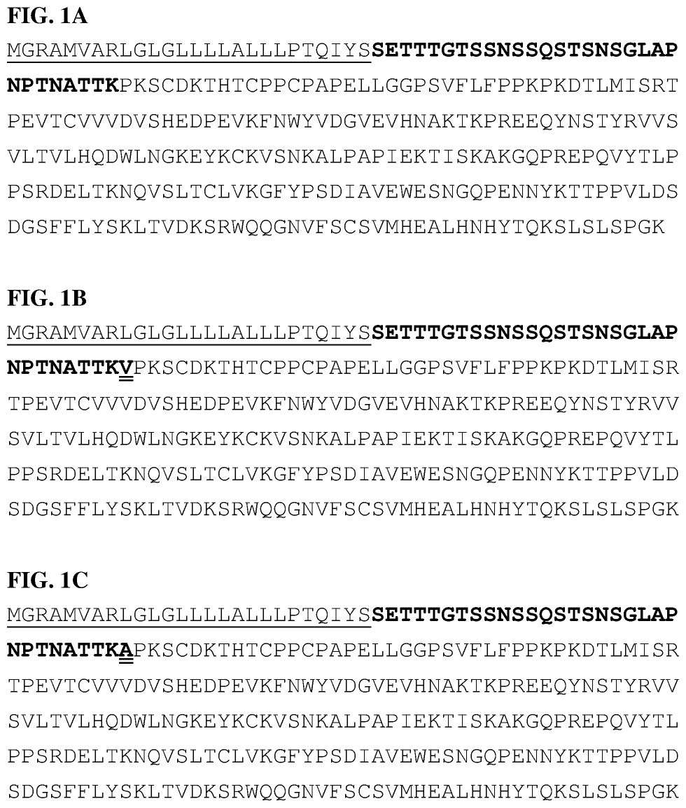

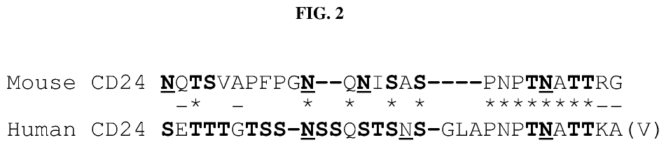

Based on preclinical research, for the construction of the fusion protein CD24Fc identified in the examples, the truncated form of native CD24 molecule of 30 amino acids, which lacks the final polymorphic amino acid before the GPI signal cleavage site (that is, a mature CD24 protein having SEQ ID NO: 2), has been used. The mature human CD24 sequence is fused to a human IgG1 Fc domain (SEQ ID NO: 7). The full length CD24Fc fusion protein is provided in SEQ ID NO: 5 (FIG. 1), and the processed version of CD24Fc fusion protein that is secreted from the cell (i.e. lacking the signal sequence which is cleaved off) is provided in SEQ ID NO: 6. Processed polymorphic variants of mature CD24 (that is, mature CD24 protein having SEQ ID NO: 1) fused to IgG1 Fc may comprise SEQ ID NO: 11 or 12.

### b. Production

The CD24 protein may be heavily glycosylated, and may be involved in functions of CD24 such as costimulation of immune cells and interaction with a damage-associated molecular pattern molecule (DAMP). The CD24 protein may be prepared using a eukaryotic expression system. The expression system may entail expression from a vector in mammalian cells, such as Chinese Hamster Ovary (CHO) cells. The system may also be a viral vector, such as a replication-defective retroviral vector that may be used to infect eukaryotic cells. The CD24 protein may also be produced from a stable cell line that expresses the CD24 protein from a vector or a portion of a vector that has been integrated into the cellular genome. The stable cell line may express the CD24 protein from an integrated replication-defective retroviral vector. The expression system may be GPEx™.

### c. Pharmaceutical Composition

The CD24 protein may be contained in a pharmaceutical composition, which may comprise a pharmaceutically acceptable amount of the CD24 protein. The pharmaceutical composition may comprise a pharmaceutically acceptable carrier. The pharmaceutical composition may comprise a solvent, which may keep the CD24 protein stable over an extended period. The solvent may be PBS, which may keep the CD24 protein stable for at least 66 months at −20° C. (−15˜-25° C.). The solvent may be capable of accommodating the CD24 protein in combination with another drug.

The pharmaceutical composition may be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The pharmaceutical composition may also be formulated as a depot preparation, which may be administered by implantation or by intramuscular injection. The composition may be formulated with suitable polymeric or hydrophobic materials (as an emulsion in an acceptable oil, for example), ion exchange resins, or as sparingly soluble derivatives (as a sparingly soluble salt, for example).

### d. Dosage

The dose of the CD24 protein may ultimately be determined through a clinical trial to determine a dose with acceptable toxicity and clinical efficacy. The initial clinical dose may be estimated through pharmacokinetics and toxicity studies in rodents and non-human primates. The dose of the CD24 protein may be 0.01 mg/kg to 1000mg/kg, and may be 1 to 500 mg/kg, depending on the desired amount of LDL-C-lowering and the route of administration. The CD24 protein may be administered by intravenous infusion or subcutaneous or intramural [that is, within the wall of a cavity or organ] injection, and the dose may be 10-1000 mg, 10-500 mg, 10-480 mg, 10-120 mg, or 10, 30, 60, 120, 240 mg, or 480 mg, where the subject is a human.

### 3. Siglec Agonists

Provided herein are agonists of Siglecs (Sialic acid-binding immunoglobulin-type lectins). Siglecs are a diverse family of cell surface proteins that bind sialic acid containing structures such as glycoproteins like CD24. Accordingly, Siglecs may have a number of different ligands and a particular sialic-acid containing ligand may bind more than one Siglec receptor. In one embodiment the Siglec agonist binds to a Siglec containing an ITIM (Immunoreceptor tyrosine-based inhibitory motif) in its cytosolic region. In another embodiment the agonist binds to a member of the human CD33-related Siglec family. In a preferred embodiment, the agonist binds to at least one of human Siglec-3, Siglec-5, Siglec-6, Siglec-7, Siglec-8, Siglec-9, Siglec-10, Siglec-11, and Siglec-12.

The Siglec agonist can be a natural Siglec ligand, such as CD24, or a portion thereof as described herein. In another embodiment, the Siglec agonist is a sialic acid-containing structure such as a glycoprotein, a glycolipid, or other sialic acid-containing structure. In yet another embodiment the Siglec agonist is an antibody that binds to the Siglec and triggers the endogenous intracellular signaling pathway mediated by the Siglec receptor.

The Siglec agonist may activate ITIM-containing Siglecs by co-inducing tyrosine phosphorylation of the ITIM domain, which results in recruitment of SHP-1 and/or SHP-2 phosphatases to Siglec or another ITIM-containing structure.

### 4. Methods of Treatment

Provided herein are methods of treating subjects with prediabetes or diabetes, or who are at risk of developing diabetes. The CD24 protein or Siglec agonist described herein may be administered to the subject, who may be in need of lowering LDL-C and/or glucose levels, which may be elevated. The subject may also be in need of treatment or prevention of atherosclerosis, or of lowering the risk of a cardiovascular disease event, which may be an atherosclerotic cardiovascular disease (ASCVD) event. The ASCVD event may be an acute coronary syndrome, myocardial infarction, stable or unstable angina, a coronary or other arterial revascularization, stroke, transient ischemic attack, or peripheral arterial disease presumed to be of atherosclerotic origin. The subject may be a mammal such as a human.

The subject may have prediabetes, and may have impaired fasting glucose (IFG) or impaired glucose tolerance (IGT). The subject may have hemoglobin A1C levels of 5.7%-6.4%, a fasting plasma glucose level of 100-125 mg/dL, or a glucose level of 140-199 mg/dL in a 2-hour post 75 g oral glucose challenge. The subject may have diabetes, and may have a fasting plasma glucose level ≥126 mg/dL, a hemoglobin A1C level ≥6.5%, or a glucose level of ≥200 mg/dL in a 2-hour 75 g oral glucose challenge.

Guidelines for diagnosing and treating elevated LDL-C levels based on a subject's characteristics are routinely used in the art. The subject may be a male or female. The subject may be of any age, but in particular may have an age of 40-75 years, or greater than 75 years. The subject may have a LDL-C greater than or equal to 70 mg/dL, 75 mg/dL, or 190 mg/dL. The subject may also be diabetic or non-diabetic, be 40-75 years old, and have a LDL-C of 70-189 mg/dL. The subject may have a 10-year ASCVD risk (defined as nonfatal myocardial infarction, coronary heart disease death, or nonfatal and fatal stroke) greater than or equal to 7.5%, or of 5-7.5%. The subject may have characteristics of a subject for whom LDL-C lowering is recommended according to the 2013 American College of Cardiology/American Heart Association Guidelines (Stone N J, et al., 2013 ACC/AHA guideline of the treatment of blood cholesterol to reduce atherosclerotic cardiovascular risk in adults: a report of the American College of Cardiology/American Heart Association Task Force on Practice Guidelines, J Am Coll Cardiol 2014; 63:2889-934). The subject may also have characteristics set forth in an update to the foregoing guidelines. The subject may have familial hypercholesterolemia, which may be caused by a mutation in the LDL receptor gene, apolipoprotein B gene, or pro-protein convertase subtilisin/kexintype 9 gene.

The subject may have been previously treated with a LDL-C-lowering drug, such as a statin. The subject may also have experienced an adverse event as a result of the drug. The adverse event may have been a muscle symptom such as pain, tenderness, stiffness, cramping, weakness, or general fatigue, and may have been a creatine phosphokinase level indicative of an increased risk for adverse muscle events (which may be >10 times the upper limit of normal). The subject may be recalcitrant to treatment with another cholesterol-lowering drug, and may have a LDL-C greater than or equal to 75 mg/dL after being treated with the other drug, which may be a statin. The subject may have graft vs. host disease, and may have exhibited a 10% or greater increase in LDL-C after having undergone a transplant in comparison to the subject's LDL-C before the transplant. The subject may have prediabetes, or an autoimmune or inflammatory disease.

### a. Administration

The route of administration of the pharmaceutical composition may be parenteral. Parenteral administration includes, but is not limited to, intravenous, intraarterial, intraperitoneal, subcutaneous, intramuscular, intrathecal, intraarticular, and direct injection. The pharmaceutical composition may be administered to a human patient, cat, dog, large animal, or an avian. The composition may be administered 1, 2, 3, 4, 5, 6, 7, 8, 9, 10, 11, or 12 times per day.

### b. Combination Treatment

The CD24 protein or Siglec agonist may be combined with another treatment such as a drug, including a statin, a bile acid-binding resin, fibrate, niacin, ezetimibe, or a drug that increases LDL receptor levels, including but not limited to an antibody or other inhibitor that antagonizes or blocks the function of PCSK9. The CD24 protein or Siglec agonist and the other drug may be administered together or sequentially.

The CD24 protein or Siglec agonist may be administered simultaneously or metronomically with other treatments. The term “simultaneous” or “simultaneously” as used herein, means that the CD24 protein or Siglec agonist and other treatment be administered within 48 hours, preferably 24 hours, more preferably 12 hours, yet more preferably 6 hours, and most preferably 3 hours or less, of each other. The term “metronomically” as used herein means the administration of the agent at times different from the other treatment and at a certain frequency relative to repeat administration.

The CD24 protein or Siglec agonist may be administered at any point prior to another treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins, 10 mins, 9 mins, 8 mins, 7 mins., 6 mins., 5 mins., 4 mins., 3 mins, 2 mins, and 1 mins. The CD24 protein or Siglec agonist may be administered at any point prior to a second treatment of the CD24 protein or Siglec agonist including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

The CD24 protein or Siglec agonist may be administered at any point after another treatment including about 1 min, 2 mins., 3 mins., 4 mins., 5 mins., 6 mins., 7 mins., 8 mins., 9 mins., 10 mins., 15 mins., 20 mins., 25 mins., 30 mins., 35 mins., 40 mins., 45 mins., 50 mins., 55 mins., 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, 48 hr, 50 hr, 52 hr, 54 hr, 56 hr, 58 hr, 60 hr, 62 hr, 64 hr, 66 hr, 68 hr, 70 hr, 72 hr, 74 hr, 76 hr, 78 hr, 80 hr, 82 hr, 84 hr, 86 hr, 88 hr, 90 hr, 92 hr, 94 hr, 96 hr, 98 hr, 100 hr, 102 hr, 104 hr, 106 hr, 108 hr, 110 hr, 112 hr, 114 hr, 116 hr, 118 hr, and 120 hr. The CD24 protein or Siglec agonist may be administered at any point prior after a previous CD24/Siglec agonist treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

### 5. Methods of Monitoring CD24 Protein Activity

The activity of the CD24 protein or Siglec agonist administered to a subject may be monitored by detecting the concentration of LDL-C or glucose or both in the subject. The subject may be undergoing treatment with the CD24 protein or Siglec agonist, such as treatment for prediabetes or an immune-mediated tissue injury, or the like. The concentration of LDL-C or glucose may be indicative of the level of CD24 protein or Siglec agonist activity in the subject, where a decrease in LDL-C or glucose in the patient indicates greater CD24 protein or Siglec agonist activity. The method may comprise obtaining a sample from the subject and detecting the amount of LDL-C or glucose in the sample. The sample may be a blood sample such as serum or plasma. Methods of measuring LDL-C and glucose concentrations are well-known in the art. For example, methods of measuring LDL-C include an ELISA based assay or a Colorimetric/Fluorometric assay following cholesterol esterase and cholesterol oxidase treatment. The amount of LDL-C may be measured by the Friedewald calculation, which may comprise calculating the amount of LDL-C based on amounts of total cholesterol, triglycerides, and high-density lipoprotein cholesterol (HDL-C) measured in the sample. The amount of HDL-C may be measured either by a precipitation procedure with dextran sulfate-Mg²⁺ or by a direct HDL-C assay. The amount of LDL-C may also be measured by the DIRECT LDL™ assay, the homogeneous N-GENEOUS™ LDL assay, or calculated LDL-C values deriving from the ApoB based equation: 0.41TC−0.32TG+1.70ApoB−0.27, (Clin Chem 1997; 43:808-815; the contents of which are incorporated herein by reference). The level of LDL-C can be monitored over time and during the course of CD24 protein or Siglec agonist treatment in order to monitor the response to treatment. As an alternative to LDL-C, the concentration of LDL particles (LDL-P) may also be measured to monitor CD24 protein or Siglec agonist activity. The LDL-P concentration may be detected directly using NMR.

The amount of CD24 protein or Siglec agonist being administered to the subject for treating an indication described herein or known in the art, may be adjusted based on the level of CD24 protein or Siglec agonist activity detected using LDL-C or glucose levels. The level of LDL-C or glucose can be monitored over a period of time or during the course of CD24 protein or Siglec agonist treatment. If the LDL-C or glucose concentration in the subject is reduced to a level within the range of normal, then the amount of CD24 protein or Siglec agonist administered to the subject may be reduced, such as by lowering the dose of CD24 protein or Siglec agonist or administering it less frequently. If the LDL-C or glucose concentration remains unchanged or remains above the range of normal, then the amount of CD24 protein or Siglec agonist administered to the subject may be increased, such as by increasing the dose of CD24 protein or or Siglec agonist administering it more frequently. Both LDL-C and glucose levels may be used in the methods of monitoring disclosed herein.

Levels of the CD24 protein or Siglec agonist administered to the subject may also be monitored, which may be by a method comprising obtaining a sample from the subject and detecting the amount of the CD24 protein or Siglec agonist in the sample. The sample may be a blood sample such serum or plasma. Protein detection methods are well-known in the art. The CD24 protein or Siglec agonist in the sample may be detected by any protein detection method, such as an immunoassay including ELISA, Gyros, MSD, Biacore, AlphaLISA, Delfia, Singulex, Luminex, Immuno-PCR, Cell-based assays, RIA, Western blot, an affinity column, and the like. The ELISA method may be sandwich ELISA or competitive ELISA. For example, the ELISA may comprise contacting the sample to an anti-CD24 protein antibody, contacting the CD24 protein-CD24 protein antibody complex with a labeled antibody that binds to the anti-CD24 protein antibody, and measuring the amount of labeled antibody by detecting a signal produced by the label, where the amount of signal correlates to the amount of CD24 protein in the sample.

The amount of CD24 protein or Siglec agonist administered to the subject may be adjusted (such as by adjusting dose and frequency of administration) based on a pharmacokinetic parameter for the CD24 protein or Siglec agonist. For example, the amount of CD24 protein administered to the subject may be adjusted to obtain a plasma CD24 concentration of greater than 1 ng/ml. In another example, the amount of CD24 protein administered to the subject is adjusted to maintain a steady state plasma concentration greater than 1 ng/mL. In another example, the amount of CD24 protein administered to the subject may be adjusted to obtain a Cₘₐₓ of the CD24 protein of at least about 1 ng/mL. In yet another example, the amount of CD24 protein administered to the subject may be adjusted to achieve a drug exposure level, as defined by the AUC_{0-inf}, of the CD24 protein of at least about 400,000 ng*hr/mL.

The present invention has multiple aspects, illustrated by the following non-limiting examples.

### EXAMPLE 1

### Soluble CD24 Proteins

FIG. 1. shows the amino acid composition of the CD24Fc fusion protein, in which the sequence of mature extracellular domain of human CD24 was fused to human IgG1 Fc. FIG. 2 shows amino acid sequence variations between mature CD24 proteins from mouse (SEQ ID NO: 3) and human (SEQ ID NO: 2). The potential O-glycosylation sites are bolded, and the N-glycosylation sites are underlined.

### EXAMPLE 2

### CD24 Pharmacokinetics in Mice

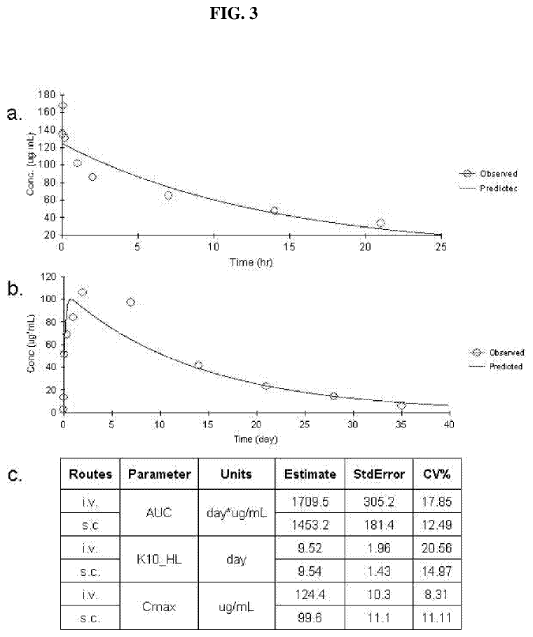

1 mg of CD24Fc (CD24Fc) was injected into naïve C57BL/6 mice and collected blood samples at different timepoints (5 min, 1 hr, 4 hrs, 24 hrs, 48 hrs, 7 days, 14 days and 21 days) with 3 mice in each timepoint. The sera were diluted 1:100 and the levels of CD24Fc was detected using a sandwich ELISA using purified anti-human CD24 (3.3 μg/ml) as the capturing antibody and peroxidase conjugated goat anti-human IgG Fc (5 μg/ml) as the detecting antibodies. As shown in FIG. 3a. The decay curve of CD24Fc revealed a typical biphase decay of the protein. The first biodistribution phase had a half-life of 12.4 hours. The second phase follows a model of first-order elimination from the central compartment. The half-life for the second phase was 9.54 days, which is similar to that of antibodies in vivo. These data suggest that the fusion protein is very stable in the blood stream. In another study in which the fusion protein was injected subcutaneously, an almost identical half-life of 9.52 days was observed (FIG. 3b). More importantly, while it took approximately 48 hours for the CD24Fc to reach peak levels in the blood, the total amount of the fusion protein in the blood, as measured by AUC, was substantially the same by either route of injection. Thus, from therapeutic point of view, different route of injection should not affect the therapeutic effect of the drug. This observation greatly simplified the experimental design for primate toxicity and clinical trials.

### EXAMPLE 3

### CD24 Lowers LDL-C Levels

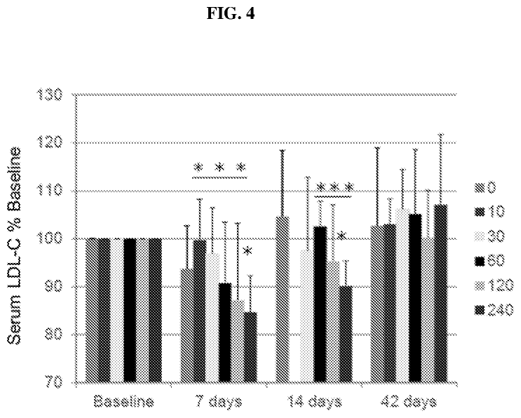

This example demonstrates that CD24 lowers LDL-C and increases leptin. Changes of fasting LDL-C in plasma from baseline were analyzed in a clinical study which is described in more detail below (see the Methods section of this example). Fasting LDL-C levels were determined among samples obtained on Day −1, Day 7, and Day 42 for Cohort 1 (CD24Fc 10 mg group). Beginning with Cohort 2 (CD24Fc 30 mg group), this lipid sampling was expanded to include Day 14. The data are summarized in Table 1. Due to an incomplete dataset in Cohort 1, Cohorts 2-5 were used to analyze for dose-dependent reduction of LDL-C levels. A statistically significant dose-dependent reduction was observed as shown in Table 1 and FIG. 4.

TABLE 1
Change in LDL-C levels on Day 7 (U1), Day 14 (U2) and Day 42 (U3) from baseline (U0, defined as 100%)

| Dose | Obs | Variable | Label | N | Mean | Std Dev | Minimum | Maximum |
|---|---|---|---|---|---|---|---|---|
| 10 mg | 6 | u0 | Baseline LDL | 6 | 100.0000000 | 0 | 100.0000000 | 100.0000000 |
| | | u1 | 7 days LDL ratio | 5 | 99.6785886 | 8.5665505 | 87.0370370 | 107.7586207 |
| | | u2 | 14 days LDL ratio | 0 | . | . | . | . |
| | | u3 | 42 days LDL ratio | 6 | 102.9957054 | 5.3134796 | 96.8085106 | 110.5769231 |
| 30 mg | 6 | u0 | Baseline LDL | 6 | 100.0000000 | 0 | 100.0000000 | 100.0000000 |
| | | u1 | 7 days LDL ratio | 6 | 96.9190313 | 9.5257894 | 86.9047619 | 113.4328358 |
| | | u2 | 14 days LDL ratio | 6 | 97.5816504 | 15.2482354 | 84.5238095 | 122.3880597 |
| | | u3 | 42 days LDL ratio | 6 | 106.1959745 | 8.2383407 | 95.2830189 | 113.4328358 |
| 60 mg | 6 | u0 | Baseline LDL | 6 | 100.0000000 | 0 | 100.0000000 | 100.0000000 |
| | | u1 | 7 days LDL ratio | 6 | 90.7620588 | 12.6697467 | 72.0720721 | 106.1728395 |
| | | u2 | 14 days LDL ratio | 6 | 102.5671170 | 5.2461286 | 96.5517241 | 110.3773585 |
| | | u3 | 42 days LDL ratio | 6 | 105.1546943 | 13.4340830 | 93.2773109 | 127.1604938 |
| 120 mg | 6 | u0 | Baseline LDL | 6 | 100.0000000 | 0 | 100.0000000 | 100.0000000 |
| | | u1 | 7 days LDL ratio | 6 | 87.1476632 | 16.0595374 | 61.7391304 | 106.4516129 |
| | | u2 | 14 days LDL ratio | 6 | 95.2625418 | 11.8341667 | 83.4782609 | 116.1290323 |
| | | u3 | 42 days LDL ratio | 6 | 100.1377165 | 9.9404474 | 87.1794872 | 112.3456790 |
| 240 mg | 6 | u0 | Baseline LDL | 6 | 100.0000000 | 0 | 100.0000000 | 100.0000000 |
| | | u1* | 7 days LDL ratio | 6 | 84.6472221 | 7.6553896 | 71.5596330 | 94.0476190 |
| | | u2* | 14 days LDL ratio | 5 | 90.1393086 | 5.2501807 | 86.2385321 | 99.0825688 |
| | | u3 | 42 days LDL ratio | 6 | 107.0369419 | 14.7154796 | 79.8449612 | 121.1009174 |
| Control | 10 | u0 | Baseline LDL | 10 | 100.0000000 | 0 | 100.0000000 | 100.0000000 |
| | | u1 | 7 days LDL ratio | 10 | 93.7350811 | 8.9747121 | 83.7837838 | 107.1428571 |
| | | u2 | 14 days LDL ratio | 8 | 104.5965396 | 13.8625952 | 83.7837838 | 125.2631579 |
| | | u3 | 42 days LDL ratio | 10 | 102.6699920 | 16.2815599 | 77.0270270 | 138.1578947 |

*P < 0.05 when compared to placebo group, student t-test.

Using cohort 1 as reference, it was determined whether CD24Fc reduced LDL-C levels in a dose- and time-dependent manner. As shown in Table 2, compared with cohort 1 which received 10 mg of CD24Fc, a significant dose-dependent reduction of LDL-C levels was observed (p<0.0001).

TABLE 2
Dose and time-dependence of LDL-C reduction in Cohorts by GEE model, using
cohort 1 (the lowest dose as reference)

| | | Standard | 95% Confidence | | | |
|---|---|---|---|---|---|---|
| Parameter | Estimate | Error | Limits | | Z | Pr > \|Z\| |
| Intercept | 98.0544 | 5.4745 | 87.3245 | 108.7842 | 17.91 | <.0001 |
| time | 1.6471 | 2.1861 | −2.6375 | 5.9317 | 0.75 | 0.4512 |
| 30 mg | 3.7167 | 7.3244 | −10.6389 | 18.0722 | 0.51 | 0.6118 |
| time* 30 mg | −1.4733 | 3.5435 | −8.4183 | 5.4718 | −0.42 | 0.6776 |
| 60 mg | −25.4898 | 14.4124 | −53.7377 | 2.7581 | −1.77 | 0.0770 |
| time* 60 mg | 10.7245 | 5.0225 | 0.8805 | 20.5685 | 2.14 | 0.0327 |
| 120 mg | −21.2684 | 9.4771 | −39.8431 | −2.6936 | −2.24 | 0.0248 |
| time* 120 mg | 6.6669 | 3.9357 | −1.0468 | 14.3806 | 1.69 | 0.0903 |
| 240 mg | −15.8681 | 6.9247 | −29.4402 | −2.2960 | −2.29 | 0.0219 |
| time* 240 mg | 5.4390 | 2.8825 | −0.2106 | 11.0887 | 1.89 | 0.0592 |

A statistically significant dose-dependent reduction of LDL-C was observed, indicating that CD24Fc is effective for lowering LDL-C in human patients.

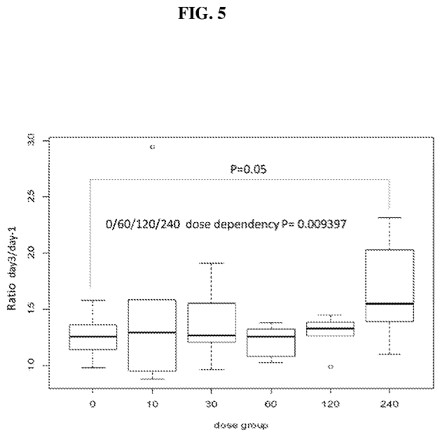

Using a Luminex bead-based immunoassay, plasma leptin levels were also measured in samples obtained on Day −1 pre-treatment and Day 3-post treatment from the 40 healthy subjects receiving CD24Fc or placebo. As shown in FIG. 5, there is a upward trend in the relative amount circulating leptin following CD24Fc treatment and between the 0, 60, 120 and 240 mg cohorts this increase is statistically significant (P=0.009397, dose-dependent general linear model regression), demonstrating a dose dependent increase above 60 mg. Furthermore, there is a statistically significant increase in the level of leptin following CD24Fc administration in the 240 mg cohort compared to placebo (0 mg) (P=0.05 as determined by Student's T test), indicating that CD24Fc is effective for increasing leptin in human patients.

### Methods

This was a Phase I, randomized, double-blind, placebo-controlled, single ascending dose study to assess the safety, tolerability, and PK of CD24Fc in healthy male and female adult subjects. A total of 40 subjects in 5 cohorts of 8 subjects each were enrolled in this study. Six of the 8 subjects in each cohort received study drug and 2 subjects received placebo (0.9% sodium chloride, saline). The first cohort was dosed with 10 mg. Succeeding cohorts received 30 mg, 60 mg, 120 mg, and 240 mg of CD24Fc or matching placebo and were dosed at least 3 weeks apart to allow for review of safety and tolerability data for each prior cohort. Administration of the next higher dose to a new cohort of subjects was permitted only if adequate safety and tolerability had been demonstrated.

In each cohort, the initial 2 subjects were 1 study drug recipient and 1 placebo recipient on Day 1. The 3rd to 5th and 6th to 8th subjects were dosed after Day 7 (a minimum of 24 hours apart between the subgroups). Each subject was dosed at least 1 hour apart in the same subgroup. If necessary, dosing of the rest of subjects was delayed pending review of any significant safety issues that may have arisen during the post-dose period involving the first or second subgroups in that cohort. The subsequent cohort was dosed at least 3 weeks after the prior cohort.

### Screening Period

The Screening Visit (Visit 1) occurred up to 21 days prior to the beginning of the active treatment period. After providing informed consent, subjects underwent screening procedures for eligibility.

### Treatment Period

Subjects were admitted to the Clinical Pharmacology Unit (CPU) on Day −1 (Visit 2), and the randomized treatment period began on Day 1 following a 10-hour minimum overnight fast. Subjects were randomly assigned to treatment with CD24Fc or placebo as a single dose. Subjects remained confined until the morning of Day 4.

### Follow-Up

All subjects returned to the CPU on Day 7, Day 14, Day 21, Day 28, and Day 42 (±1 day) for follow-up visits (Visit 3, Visit 4, Visit 5, Visit 6, and Visit 7). Visit 7 was the final visit for all subjects.

Duration of Treatment: The total study duration for each subject was up to 63 days. Single-dose administration occurred on Day 1.

### Number of Subjects

Planned: 40 subjects

Screened: 224 subjects

Randomized: 40 subjects

Completed: 39 subjects

Discontinued: 1 subject

Diagnosis and Main Criteria for Inclusion: The population for this study was healthy males and females between the ages of 18 and 55 years, inclusive, with a body mass index between 18 kg/m² and 30 kg/m², inclusive.

### Investigational Product and Comparator Information

CD24Fc: single dose of 10 mg, 30 mg, 60 mg, 120 mg, or 240 mg administered via IV infusion; lot number: 09MM-036. CD24Fc was a fully humanized fusion protein consisting of the mature sequence of human CD24 and the fragment crystallizable region of human immunoglobulin G1 (IgG1Fc). CD24Fc was supplied as a sterile, clear, colorless, preservative-free, aqueous solution for IV administration. CD24Fc was formulated as single dose injection solution, at a concentration of 10 mg/mL and a pH of 7.2. Each CD24Fc vial contained 160 mg of CD24Fc, 5.3 mg of sodium chloride, 32.6 mg of sodium phosphate dibasic heptahydrate, and 140 mg of sodium phosphate monobasic monohydrate in 16 mL±0.2 mL of CD24Fc. CD24Fc was supplied in clear borosilicate glass vials with chlorobutyl rubber stoppers and aluminum flip-off seals.

Matching placebo (0.9% sodium chloride, saline) administered via IV infusion; lot numbers: P296855, P311852, P300715, P315952.

The intent-to-treat (ITT) Population consisted of all subjects who received at least 1 dose of the study drug. The ITT Population was the primary analysis population for subject information and safety evaluation.

Clinical laboratory evaluations (chemistry, hematology, and urinalysis) were summarized by treatment and visit. Change from baseline was also summarized. Vital signs (blood pressure, heart rate, respiratory rate, and temperature) were summarized by treatment and time point. Change from baseline was also summarized. All physical examination data were listed. Electrocardiogram parameters and the change from baseline were summarized. Overall interpretations were listed. Fasting LDL-C and high density lipoprotein cholesterol were obtained on Day −1, Day 7, and Day 42 for Cohort 1 (CD24Fc 10 mg group). Beginning with Cohort 2 (Cd24Fc 30 mg group), this lipid sampling was expanded to include Day 14.

### EXAMPLE 4

### CD24 Pharmacokinetics in Humans

This example shows an analysis of the pharmacokinetics of a CD24 protein in humans.

### Plasma CD24Fc Concentration

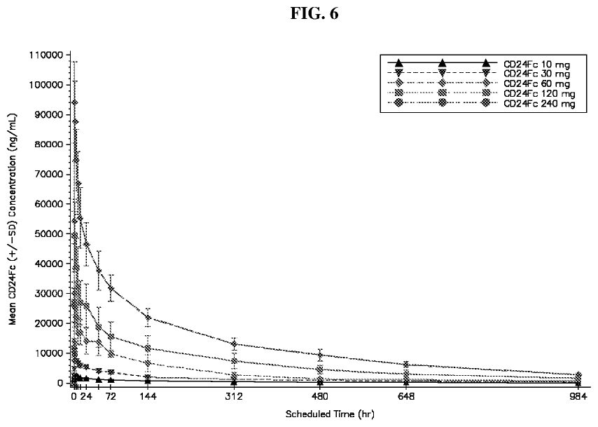

As shown in FIG. 6, the mean plasma concentration of CD24Fc increased proportionally to the dose of CD24Fc administered. For all dose groups except 120 mg, the maximum mean plasma concentration of CD24Fc was reached at 1 hour post-dose. The maximum mean plasma concentration of CD24Fc for the 120 mg group was reached at 2 hours post-dose. By Day 42 (984 hours), the mean plasma concentration of CD24Fc for all groups had decreased to between 2% and 4% of the maximum mean plasma concentration.

Table 3 summarizes the plasma CD24Fc PK parameters by treatment for the PK Evaluable Population.

TABLE 3
Summary of Plasma CD24Fc Pharmacokinetic Parameters by Treatment—PK
Evaluable Population

| | CD24Fc | CD24Fc | CD24Fc | CD24Fc | CD24Fc |
|---|---|---|---|---|---|
| Parameter | 10 mg | 30 mg | 60 mg | 120 mg | 240 mg |
| Statistic | (N = 6) | (N = 6) | (N = 6) | (N = 6) | (N = 6) |
| Cₘₐₓ (ng/mL) | | | | | |
| n | 6 | 6 | 6 | 6 | 6 |
| Mean (SD) | 2495 | 9735 | 30 083 | 52 435 | 95 865 |
| | (576) | (1715) | (7179) | (9910) | (10 734) |
| CV % | 23.1 | 17.6 | 23.9 | 18.9 | 11.2 |
| Median | 2371 | 9218 | 29 026 | 50 401 | 93 206 |
| Min, Max | 1,967, | 8,583, | 22,557, | 40,434, | 81,296, |
| | 3,390 | 13,086 | 42,628 | 65,704 | 110,110 |
| Geometric mean | 2,442 | 9,625 | 29,424 | 51,666 | 95,365 |
| Geometric CV% | 22.8 | 16.1 | 23.0 | 19.0 | 11.2 |
| AUC_{0-42d} (ng*hr/mL) | | | | | |
| n | 6 | 6 | 6 | 6 | 6 |
| Mean (SD) | 423,061 | 1,282,430 | 3,226,255 | 6,541,501 | 12,704,705 |
| | (99,615) | (88,798) | (702,862) | (2,190,944) | (1,918,596) |
| CV % | 23.5 | 6.9 | 21.8 | 33.5 | 15.1 |
| Median | 434,043 | 1,302,719 | 3,124,933 | 5,785,142 | 12,563,426 |
| Min, Max | 291,020, | 1,175,733, | 2,487,550, | 4,485,193, | 10,466,635, |
| | 528,079 | 1,403,024 | 4,139,748 | 9,415,266 | 15,693,606 |
| Geometric mean | 412,795 | 1,279,851 | 3,163,252 | 6,249,552 | 12,586,731 |
| Geometric CV % | 25.0 | 7.0 | 22.0 | 33.8 | 15.0 |
| AUC_{0-inf} (ng*hr/mL) | | | | | |
| n | 6 | 6 | 6 | 6 | 6 |
| Mean (SD) | 462,260 | 1,434,464 | 3,497,196 | 7,198,196 | 13,861,796 |
| | (116,040) | (131,316) | (705,653) | (2,458,320) | (1,962,780) |
| CV % | 25.1 | 9.2 | 20.2 | 34.2 | 14.2 |
| Median | 470,426 | 1,422,205 | 3,519,732 | 6,463,665 | 13,713,034 |
| Min, Max | 310,956, | 1,281,715, | 2,703,655, | 4,910,640, | 11,822,988, |
| | 596,599 | 1,650,503 | 4,309,023 | 10,479,940 | 17,175,236 |
| Geometric mean | 449,583 | 1,429,578 | 3,437,036 | 6,862,129 | 13,750,972 |
| Geometric CV % | 26.7 | 9.0 | 20.7 | 34.6 | 13.8 |
| Tₘₐₓ (hr) | | | | | |
| n | 6 | 6 | 6 | 6 | 6 |
| Mean (SD) | 1.15 (0.42) | 1.17 (0.41) | 1.01 (0.01) | 1.34 (0.51) | 1.33 (0.52) |
| CV % | 36.1 | 35.0 | 1.2 | 38.0 | 38.7 |
| Median | 1.00 | 1.00 | 1.00 | 1.03 | 1.00 |
| Min, Max | 0.92, 2.00 | 1.00, 2.00 | 1.00, 1.03 | 1.00, 2.00 | 1.00, 2.00 |
| t½ (hr) | | | | | |
| n | 6 | 6 | 6 | 6 | 6 |
| Mean (SD) | 280.83 | 327.10 | 279.82 | 286.45 | 285.33 |
| | (22.37) | (41.32) | (65.59) | (23.38) | (24.33) |
| CV % | 8.0 | 12.6 | 23.4 | 8.2 | 8.5 |
| Median | 279.61 | 317.23 | 264.69 | 290.76 | 287.74 |
| Min, Max | 258.87, 321.26 | 289.82, 394.24 | 210.18, 362.46 | 243.89, 309.26 | 249.24, 322.26 |
| AUCextr (%) | | | | | |
| n | 6 | 6 | 6 | 6 | 6 |
| Mean (SD) | 7.61 (2.14) | 10.44 (2.94) | 7.88 (4.26) | 8.92 (1.94) | 8.46 (1.99) |
| CV % | 28.1 | 28.2 | 54.0 | 21.8 | 23.5 |
| Median | 7.16 | 10.01 | 6.35 | 9.27 | 8.45 |
| Min, Max | 5.46, 11.47 | 7.10, 15.05 | 3.92, 14.48 | 5.49, 10.99 | 5.56, 11.50 |
| CL (L/hr) | | | | | |
| n | 6 | 6 | 6 | 6 | 6 |
| Mean (SD) | 0.0229 | 0.0211 | 0.0178 | 0.0183 | 0.0176 |
| | (0.0061) | (0.0019) | (0.0036) | (0.0058) | (0.0023) |
| CV % | 26.7 | 8.8 | 20.5 | 31.7 | 13.3 |
| Median | 0.0216 | 0.0211 | 0.0173 | 0.0191 | 0.0175 |
| Min, Max | 0.0168, 0.0322 | 0.0182, 0.0234 | 0.0139, 0.0222 | 0.0115, 0.0244 | 0.0140, 0.0203 |
| Vd (L) | | | | | |
| n | 6 | 6 | 6 | 6 | 6 |
| Mean (SD) | 9.153 | 9.867 | 7.289 | 7.491 | 7.276 |
| | (1.943) | (0.804) | (2.592) | (2.202) | (1.426) |
| CV % | 21.2 | 8.1 | 35.6 | 29.4 | 19.6 |
| Median | 8.507 | 10.007 | 7.486 | 7.691 | 7.151 |
| Min, Max | 7.326, 12.010 | 8.771, 10.958 | 4.222, 11.139 | 4.933, 9.974 | 5.814, 9.438 |

AUC_{0-42d} = area under the concentration-time curve from time 0 to 42 days;
AUC_{0-inf} = area under the concentration-time curve extrapolated from time 0 to infinity;
AUCextr = percentage of AUC_{0-inf} that was due to extrapolation from the time of the last measurable concentration, per subject, to infinity;
CL = total body clearance;
Cₘₐₓ = maximum observed plasma drug concentration;
CV % = coefficient of variation;
Min = minimum; Max = maximum;
SD = standard deviation;
t½ = terminal elimination half-life;
Tₘₐₓ = time of maximum observed plasma drug concentration;
V_{d} = volume of distribution.

### Plasma CD24Fc Dose Proportionality Analysis

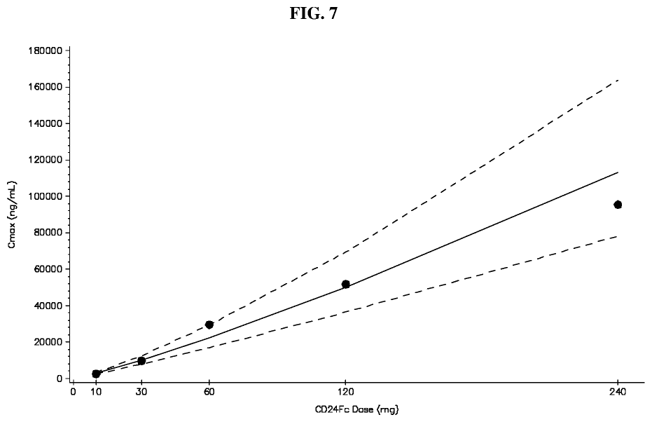

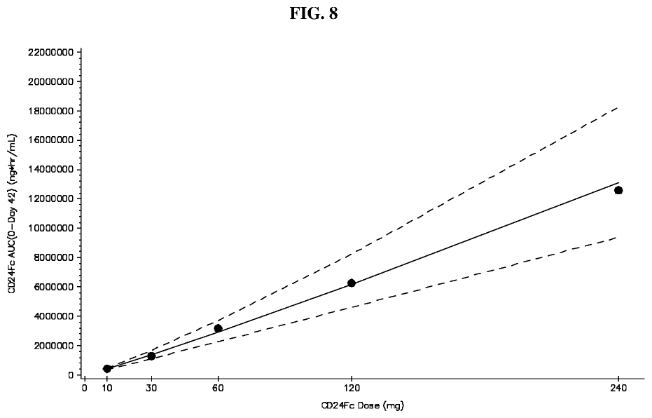

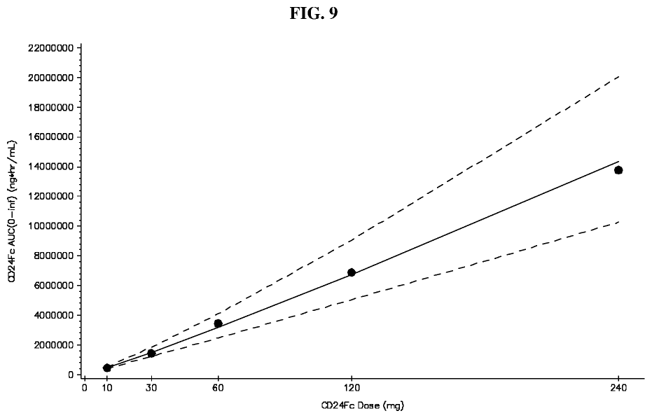

FIG. 7 shows a dose proportionality plot of CD24Fc Cₘₐₓ versus dose for the PK Evaluable Population. FIG. 8 shows a dose proportionality plot of CD24Fc AUC_{0-42d} versus dose for the PK Evaluable Population. FIG. 9 shows a dose proportionality plot of CD24Fc AUC_{0-inf} versus dose for the PK Evaluable Population. Table 4 shows a power analysis of dose proportionality.

TABLE 4
Power Analysis of Dose Proportionality: Plasma CD24Fc Pharmacokinetic Parameters-PK Evaluable Population

| | CD24Fc | CD24Fc | CD24Fc | CD24Fc | CD24Fc | Dose Proportionality | | |
|---|---|---|---|---|---|---|---|---|
| Parameter | 10 mg | 30 mg | 60 mg | 120 mg | 240 mg | Slope | Standard | |
| Statistic | (N = 6) | (N = 6) | (N = 6) | (N = 6) | (N = 6) | Estimate | Error | 90% CI |
| Cₘₐₓ (ng/mL) | | | | | | 1.172 | 0.040 | (1.105, 1.240) |
| Geometric mean | 2,441.8 | 9,624.9 | 29,424.4 | 51,666.4 | 95,364.9 | | | |
| Geometric CV % | 22.8 | 16.1 | 23.0 | 19.0 | 11.2 | | | |
| AUC_{0-42d} (ng*hr/mL) | | | | | | 1.088 | 0.036 | (1.027, 1.148) |
| Geometric mean | 412,794.8 | 1,279,850.8 | 3,163,251.7 | 6,249,551.9 | 12,586,731.3 | | | |
| Geometric CV % | 25.0 | 7.0 | 22.0 | 33.8 | 15.0 | | | |
| AUC_{0-inf} (ng*hr/mL) | | | | | | 1.087 | 0.036 | (1.026, 1.148) |
| Geometric mean | 449,583.5 | 1,429,577.5 | 3,437,035.6 | 6,862,128.7 | 13,750,972.4 | | | |
| Geometric CV % | 26.7 | 9.0 | 20.7 | 34.6 | 13.8 | | | |

Geometric CV % = 100*sqrt(exp(SD²) − 1), where SD was the standard deviation of the log-transformed data. The power model was fitted by restricted maximum likelihood, regressing the log-transformed PK parameter on log transformed dose. Both the intercept and slope were fitted as fixed effects. Dose proportionality was not rejected if the 90% CI lies within (0.8, 1.25).
AUC_{0-42d} = area under the concentration-time curve from time 0 to 42 days;
AUC_{0-inf} = area under the concentration-time curve extrapolated from time 0 to infinity;
CI = confidence interval;
Cₘₐₓ = maximum observed plasma drug concentration;
CV % = coefficient of variation;
PK = pharmacokinetic;
SD = standard deviation.

The Cₘₐₓ slope estimate was 1.172 with a 90% CI of 1.105 to 1.240. The AUC_{0-42d} lope estimate was 1.088 with a 90% CI of 1.027 to 1.148. The AUC_{0-inf} slope estimate was 1.087 with a 90% CI of 1.026 to 1.1.

### Pharmacokinetic Conclusions

The Cₘₐₓ and AUCs of plasma CD24Fc increased proportionally to the doses administered in mouse, monkey and human. The plasma CD24Fc reached Tₘₐₓ between 1.01 and 1.34 hours. The t_{1/2} of plasma CD24Fc ranged between 280.83 and 327.10 hours.

### EXAMPLE 5

### CD24Fc Reduces LDL-C Levels Among HCT Patients

To confirm the effect of CD24Fc on LDL-C levels, the effect of CD24Fc on LDL-C levels in hematopoietic cell transplantation (HCT) patients was prospectively tested. This Phase IIa trial (ClinicalTrials.gov Identifier: NCT02663622) was a randomized double blind trial comprising two single ascending dose cohorts (240 mg and 480 mg) and a single multi-dose cohort (480 mg (day −1), 240 mg (day +14) and 240 mg (day +28)) of CD24Fc in addition to SOC for the prevention of acute graft-versus-host disease (GVHD) following myeloablative allogeneic hematopoietic cell transplantation.

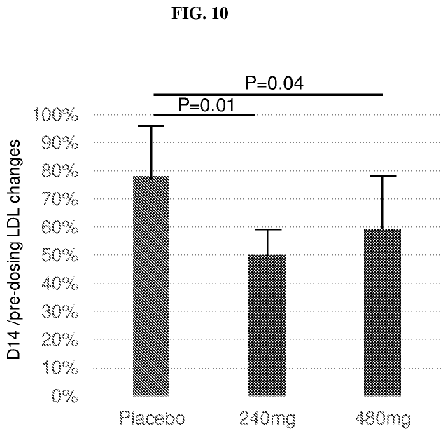

As shown in FIG. 10, at 15 days after doing of placebo, HCT patients had approximately 80% of the pre-dosing levels of LDL-C. This level was reduced to 50% and 60%, respectively, among patients receiving 240 mg (P=0.01) or 480 mg (P=0.04). The significant reductions confirm the activity of CD24Fc in reducing LDL-C in human.

### EXAMPLE 6

### CD24Fc Improves Glucose and Lipid Homeostasis in Human and Mice

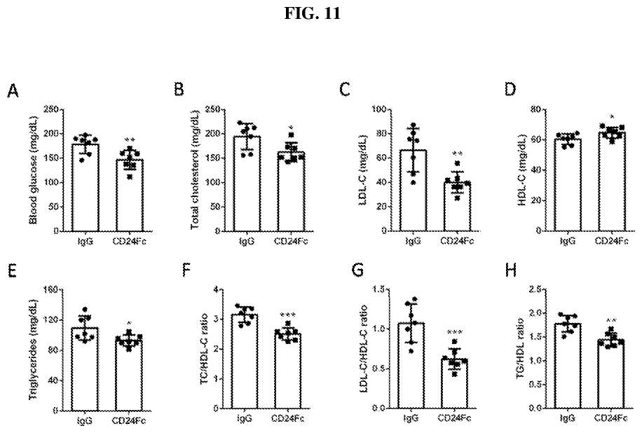

To substantiate the reductions in LDL-C observed with clinical samples, the effects of CD24Fc were tested in a diet-induced obese (DIO) mouse model. As shown in FIG. 11A, CD24Fc significantly reduced blood glucose levels under fasting conditions. In addition, CD24Fc significantly decreased total cholesterol (TC), triglycerides (TG) and low-density lipoprotein cholesterol (LDL-C) levels, while increasing high-density lipoprotein cholesterol (HDL-C) levels (FIGS. 11B-E). The ratio of TC/HDL, LDL/HDL and TG/HDL also decreased after CD24Fc treatment (FIG. 11F-H). Thus, CD24Fc improves glucose and lipid homeostasis in human and mice.

### EXAMPLE 7

### CD24Fc Interacts with Siglecs and Induces Association Between SHP-1 and Siglecs G and E

It has been previously demonstrated that CD24 binds to at least 3 different lectins with different functions. First, CD24 binds to Galectin-3 that, like other Galectins, recognizes galactose-containing saccharide structures. Galectin-3 has been shown to be involved in a variety of biological processes and, as a result, is implicated in a number of disease indications, including inflammation. Secondly, CD24 may negatively regulate host response to tissue damage-associated molecular pattern (DAMP) through its interaction with Sialic acid binding Ig-like lectin 10 (Siglec 10). We have also reported that CD24 binds to several DAMPs directly, which may enhance its activity through Siglec G/10 as described below. Thirdly, CD24 binds to myelin associated glycoprotein (MAG), which inhibits neuron regeneration and neurite growth. Therefore, by interacting with these endogenous proteins, CD24Fc may inhibit inflammation and autoimmunity while promote neuro-regeneration.

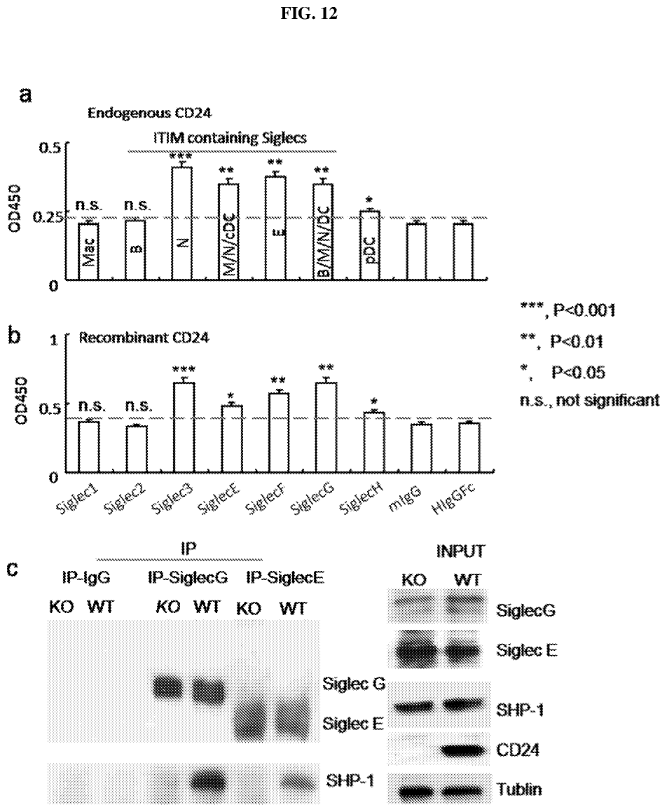

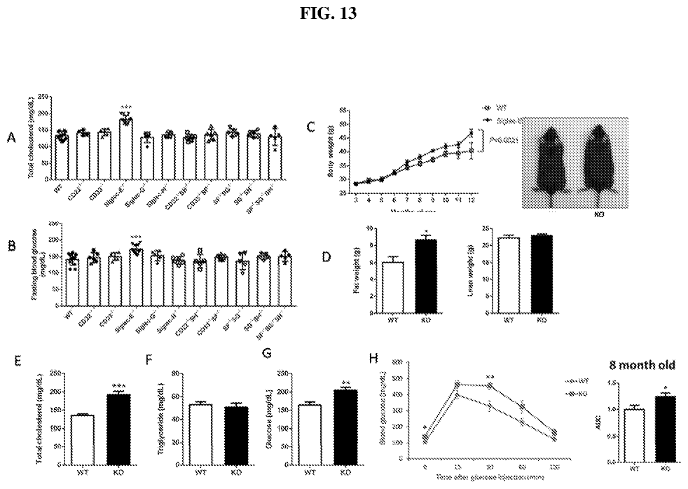

To further assess the specificity of the CD24-Siglec interaction, fusion proteins for all ITIM-containing and two non-ITIM-containing Siglecs were expressed and assayed to see whether these Siglecs could bind to CD24 expressed in spleen cells. As shown in FIG. 12a, whereas Siglecs 1 and 2 did not capture endogenously expressed CD24 from spleen cell lysate, a significant interaction was observed between CD24 and Siglecs E, F, G, and 3. The interaction is direct as recombinant CD24Fc interacts with recombinant Siglecs in the absence of any other cellular products (FIG. 12b). An important question is whether CD24 stimulates Siglecs under physiological conditions. This could be addressed by testing the engagement of endogenous Siglecs by endogenous CD24. Because SHP-1 associates with all ITIM-containing Siglecs tested and this association is inducible by Siglec ligation, its association with Siglec was used as a marker for endogenous stimulation. Siglec G and E were chosen because they are broadly expressed in major innate responder cells. Siglec G or Siglec E were precipitated from WT and CD24-deficient spleen cell lysate and Western blot was used to determine the amount of co-immunoprecipitated SHP-1. As shown in FIG. 12c, while high levels of SHP-1 associated with Siglec G and E from WT spleen cells, very little association was observed in CD24^{-/-} spleen cells lysate. This difference can be attributed to CD24 expression and not variations in Siglec or SHP-1 levels (FIG. 12c, right panels).

### EXAMPLE 8

### Identification of CD24Fc Receptors in Regulation of Metabolic Disorders

As the first test to identify a Siglec receptor potentially responsible for the negative regulation of metabolic disorders, it was determined whether the absence of Siglecs would cause metabolic disorders. Thus, mice were generated with single and combined deletions in Siglecs using the CRISPR/Cas9 system, and then used in metabolic studies.

As shown in FIGS. 13A and 13B, under normal diet, Siglece mutant mice had higher fasting blood glucose and total cholesterol levels than WT controls. In contrast no other single or double mutants (CD22 KO, CD33 KO, Siglec-G KO, Siglec-H KO, CD22/Siglec-H KO, CD33/Siglec-F KO, Siglec-F/Siglec-G KO, Siglec-G/Siglec-H KO, Siglec-F/Siglec-G/Siglec-H KO) had a significant impact on their cholesterol and fasting glucose levels. To investigate whether Siglec-E deficiency leads to alterations in systemic metabolic homeostasis, body weight, glucose metabolism, and lipid levels were examined in knockout mice on a normal diet. Weight gain (FIG. 13C) and fat content (FIG. 13D) in Siglec-E KO mice were significantly higher than WT controls as chow-fed mice age (FIGS. 13C and 13D). Siglec-E KO mice also had higher total cholesterol levels and fasting blood glucose (FIG. 13G), but there was no significant difference in triglycerides (FIG. 13F). Additionally, in these older chow-fed mice, Siglec-E KO mice exhibited defects in a glucose tolerance test (FIG. 13H).

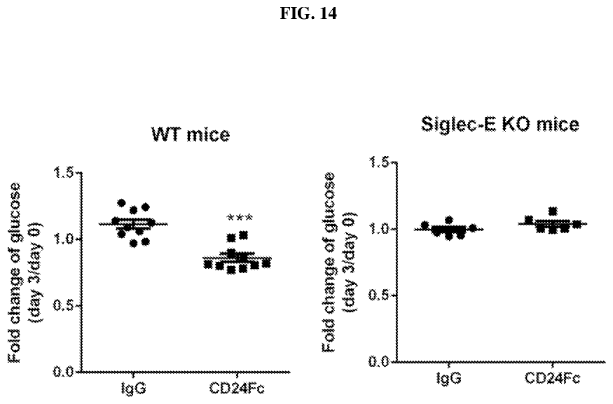

Since Siglec E interacts with CD24Fc and regulates lipid and glucose metabolism in mice, it is intriguing that CD24Fc may exert its therapeutic effect through interacting with Siglec E. To test this hypothesis, WT and Siglece KO mice were first treated after 3 months of high fat-feeding. As shown in FIG. 14, a single injection of CD24Fc significantly reduced fasting glucose levels in 3 days in WT, but not Siglec E-deficient mice. Therefore, Siglec E is necessary for the therapeutic effect of CD24Fc.

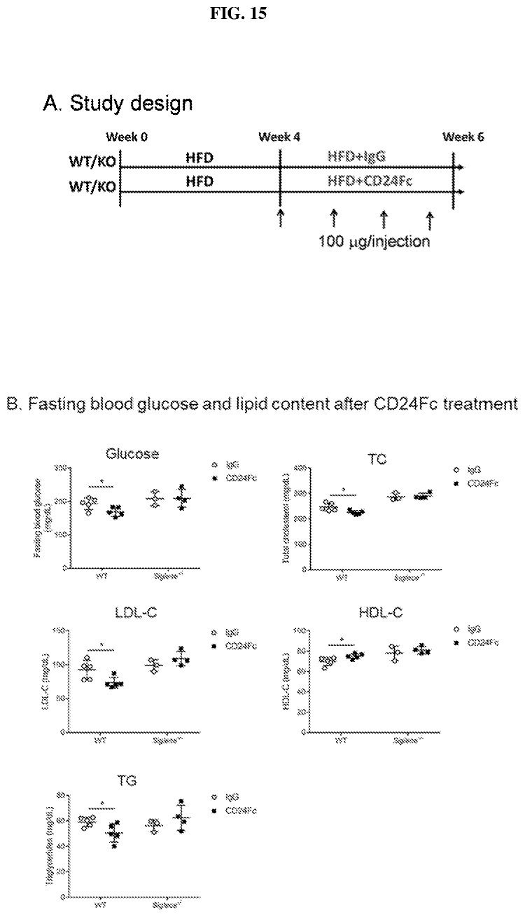

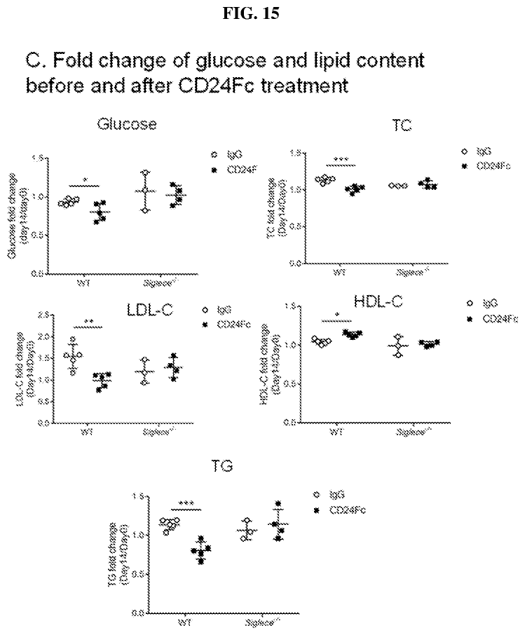

In a second experiment, WT and Siglece KO mice were treated after being fed with HFD for 4 weeks starting at the age of 8 weeks old. Mice were then injected intraperitoneally with CD24Fc (100 μg per dose) or an equivalent amount of isotype control IgG twice a week for 2 weeks (schematic shown in FIG. 15A). As shown in FIG. 15B and C, glucose, total cholesterol, LDL-C and total glycerides were decreased in WT mice following CD24Fc treatment, but not in Siglec E-deficient mice. Furthermore, HDL-C demonstrated a corresponding increase in CD24Fc treated mice with no effect in Siglec E-deficient mice.

### EXAMPLE 9

### CD24Fc Stimulates Siglec E to Reduce Fatty Acid-Induced Inflammatory Response by Macrophages

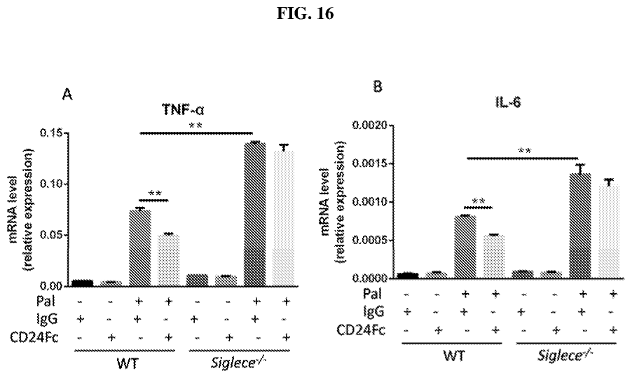

Inflammatory response to free fatty acids by macrophages plays an important role in metabolic disorders. To determine the function of the CD24-Siglec-E axis under metabolic stress, peritoneal macrophages were isolated from CD24 KO, Siglec-E KO and WT mice, and treated with palmitic fatty acids, which are elevated in obesity due to increased release from adipose tissue. As shown in FIG. 16, palmitic fatty acid stimulation induced mRNA expression and protein production of TNF-α and IL-6 in WT macrophages, and these responses were significantly reduced in the presence of CD24Fc. However, Siglec &KO macrophages were not responsive to CD24Fc. These data demonstrate that Siglec E is necessary for CD24Fc-mediated suppression of inflammatory response by macrophages.

### EXAMPLE 10

### CD24Fc Alleviates Obesity-Related Metabolic Disorders in Glucose Metabolism in DIO Mice

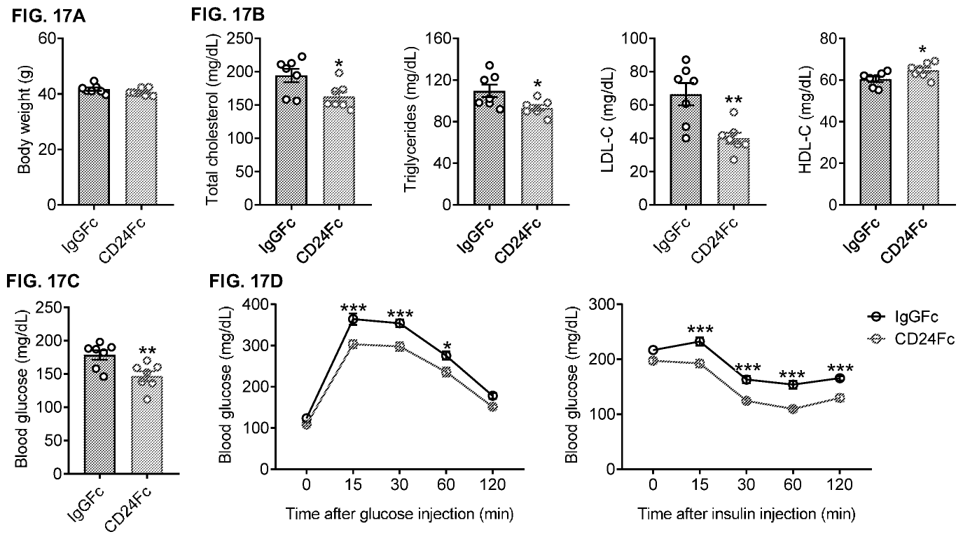

To test the therapeutic effect in obese mice, we administered DIO mice with CD24Fc or IgGFc control for 4 weeks after obesity was established and then tested the metabolic parameters. In the absence of any impact on weight gain in the short treatment window, CD24Fc therapy improved fasting glucose and lipid profiles (FIGS. 17A-C). GTT and ITT tests also revealed a significant improvement in glucose tolerance and insulin sensitivity in CD24Fc-treated DIO mice (FIG. 17D).

### Methods

Mice and diets: Cd24^{-/-}, Siglecg and Siglece^{-/-} C57BL/6 mice have been described (Chen et al., 2014; Nielsen et al., 1997). All strains were backcrossed with C57BL/6 mice for 6 or more generations. We used age- and sex-matched littermates or wild type C57BL/6 mice as controls. Leptin-deficient (ob/ob) mice were purchased from The Jackson Laboratory. All the mice were maintained at constant temperature (23±2° C.) with a 12-hour light/12-hour dark cycle and given free access to food and water prior to our study. For metabolic studies, male mice were fed with HFD consisting of 60% of calories from fat (D12492, Research Diets Inc.) starting at 8-10 weeks of age for 12 weeks. Mouse body weight and food intake were measured every week.

CD24Fc protein therapeutic studies in DIO mice: WT, Siglece or ob/ob mice were injected intraperitoneally with CD24Fc (100 μg, per dose, Oncolmmune Inc.) or an equivalent amount of control IgGFc twice a week. Fasting blood glucose and lipid profiles were detected after CD24Fc or IgG treatment. For the prevention groups, CD24Fc administration was begun concurrently with HFD feeding at 8 weeks of age for 8 weeks. For the therapy groups, CD24Fc treatment was performed in mice with established obesity (8 weeks of HFD) for 4 more weeks.

Tissue processing and histological analyses: After HFD treatment, DIO mice were anesthetized with isoflurane. Representative images of their physical appearance were taken and body composition was detected by dual energy X-ray absorptiometry (DEXA). The mice were then euthanized, livers, white adipose and brown adipose tissues were immediately harvested, photographed and weighed. For histology, the tissues were fixed in 10% formalin and embedded in paraffin. The tissues were then cut into 5 μm sections and stained with hematoxylin-eosin (H&E). Liver sections were stained with Mason's Trichrome for fibrosis studies.

Metabolic studies: For the glucose tolerance tests (GTTs), mice were injected intraperitoneally with 1 g/kg glucose (Sigma) after 12 hrs of fasting. Blood glucose levels were measured at 0, 15, 30, 60 and 120 min from tail blood using the One Touch Ultra glucometer (Lifescan). For the insulin tolerance tests (ITTs), an intraperitoneal injection of 1 U/kg insulin (Sigma) was given to mice after 6 hrs of fasting. Blood glucose levels were determined as described above. The serum TC, TG, HDL-C, LDL-C and NEFA levels were measured with commercial kits (Randox). Serum cytokines were determined using mouse cytokine bead array designed for inflammatory cytokines (BD Biosciences).

Insulin sensitivity study: For examination of in vivo insulin signaling, mice were fasted overnight and followed with an intraperitoneal injection of insulin (1 U/kg). Liver were harvested and snap-frozen in RIPA buffer 10 min after injection for phospho-Akt analysis.

Macrophages culture and stimulation: Peritoneal macrophages from WT, Cd24^{-/-} and Siglece^{-/-} mice were isolated 3 days after intraperitoneal injection of 3% thioglycollate (Sigma). The cells were plated in 6-well plates at a density of 1.2×106 cells/well and cultured in RPMI medium containing 10% fetal bovine serum (FBS). The cells were then stimulated with palmitate-bovine serum albumin (BSA) or unmodified BSA control (500 μM) for 16 h. For CD24Fc treatment studies, peritoneal macrophages from WT and Siglece^{-/-} mice were challenged with palmitate-BSA or BSA control (500 μM) and concurrently treated with CD24Fc (10 μg/ml) or IgG control for 16 hours. Supernatant and cell lysate were collected for ELISA, immunoblot and gene expression analysis. Palmitate (Sigma) was conjugated with BSA before treatment. Palmitate was dissolved in 95% ethanol at 60° C. and prepared as a 50 mM solution. The palmitate solution was then diluted with RPMI medium containing 1% BSA to obtain the 500 μM palmitate concentration.

RNA extraction and Real-time PCR analysis: Total RNA was isolated from tissues and cells using TRIzol reagent (Invitrogen). For reverse transcription, cDNA was synthesized from RNA samples with a Superscript First-Strand Synthesis System (Invitrogen). Quantitative real-time PCR was performed with SYBR Green PCR Master Mix (Applied Biosystems) using the Applied Biosystems 7500 Real-time PCR System according to the manufacturer's instructions. Gene expression levels were calculated after normalization to the housekeeping gene β-actin or GAPDH. Western blot: Tissues and cells were lysed with RIPA lysis buffer (Thermo) containing protease inhibitor (Sigma) and phosphatase inhibitor (Sigma). Total protein was quantified by BCA assay (Thermo). Equal amounts of each protein sample were electrophoresed on NuPAGE 4-12% Bis-Tris Protein Gels (Life Technologies) and transferred to PVDF membranes (Millipore). Individual proteins were determined with the specific antibodies and actin was used as an internal loading control.

Immunoprecipitation: The spleens of the indicated mice (8-10 weeks) were collected, sliced and pressed through the strainer to get single cells. The red blood cells were removed using the ACK buffer (Thermo). Then the spleen cell lysates were prepared in the buffer B (1% Triton X-100, 150 mM NaCl, 3 mM MnCl2, 1 mM CaCl2, 1 mM MgCl2, 25 mM Tris-HCl, pH 7.6) with protease inhibitor cocktail (Sigma) for immunoprecipitation or western blot. For immunoprecipitation, cell lysates were pre-cleared with Protein A/G-conjugated agarose beads (Santa Cruz) at 4° C. for 2 hours with rotation, then incubated with anti-CD24 antibody (M1/69, Biolegend) or control Rat anti-IgG (Santa Cruz) overnight at 4° C. The cell lysates were then incubated with Protein A/G-conjugated agarose beads for an additional 2 hours. The beads were washed four times with buffer B and re-suspended in SDS sample buffer (non-reducing condition) for western blot analysis.

Immunofluorescence: For immunofluorescence staining, livers were embedded in OCT compound and frozen at −80° C. The tissues were then cut into 7 μm sections using a cryostat. For peritoneal macrophages, cells were seeded on chamber slides (Thermo). The slides were washed in PBS, fixed in 4% fresh paraformaldehyde for 15 min, permeabilized with 0.5% Triton X-100 in PBS for 5 min and blocked with 3% BSA in PBS for 60 min at room temperature. The slides were then stained with NF-κB/p65 antibody (Cell Signaling Technology) in PBS overnight at 4° C. After washing with PBST for 3 times, the slides were incubated with Alexa Fluor 594-conjugated goat anti-rabbit (Life technology) for 60 min at room temperature. Nuclei were stained with DAPI for 5 min. Fluorescent images were obtained using a fluorescent microscope.

Statistical analysis: The specific tests used to analyze each set of experiments are indicated in the figure legends. Data were analyzed using an unpaired two-tailed Student's t test to compare between two groups, one-way analysis of variance (ANOVA) for multiple comparisons, two-way ANOVA for body weight, GTT and ITT data that were repeatedly measured. All statistical tests were performed using GraphPad Prism (GraphPad Software, San Diego, Calif.), and P<0.05 was considered statistically significant.

1. A method of treating a subject in need thereof with diabetes, prediabetes or at risk of developing diabetes, comprising administering a CD24 protein to the subject.

2. The method of claim 1, wherein the subject is in need of reducing serum LDL-C levels.

3. The method of claim 1, wherein the subject is in need of for reducing blood glucose levels.

4. The method of claim 1, wherein the subject is in need of treatment for cardiovascular disease (CVD), or reducing the risk of CVD, diabetes or an atherosclerotic CVD event.

5. The method of claim 1, wherein the subject has impaired fasting glucose or impaired glucose tolerance.

6. The method of claim 1, where the subject has insulin insensitivity.

7. The method of claim 5, wherein the subject has at least one of a hemoglobin A1C level of 5.7-6.4%, a fasting plasma glucose level of 100-125 mg/dL, and a glucose level of 140-199 mg/dL in a 2-hour post 75 g oral glucose challenge.

8. The method of claim 1, wherein the subject has diabetes.

9. The method of claim 8, wherein the subject has at least one of a fasting plasma glucose level of ≥126 mg/dL, a hemoglobin A1C level of ≥6.5%, and a glucose level of ≥200 mg/dL in a 2-hour 75 g oral glucose challenge.

10. The method of claim 1, wherein the subject has an elevated LDL-C level.

11. The method of claim 10, wherein the subject has a LDL-C level greater than or equal to 75 mg/dL.

12. The method of claim 10, wherein the subject has a LDL-C level of greater than or equal to 70 mg/dL or 190 mg/dL.

13. The method of claim 2, wherein the subject has been previously treated with another LDL-C-lowering drug, wherein the other LDL-C-lowering drug is not a CD24 protein.

14. The method of claim 13, wherein the other LDL-C-lowering drug is a statin.

15. The method of claim 13, wherein the other LDL-C-lowering drug is an antagonist of PCSK9.

16. The method of claim 1, wherein the CD24 protein comprises a mature human CD24 polypeptide or a variant thereof.

17. The method of claim 16, wherein the mature human CD24 polypeptide comprises the sequence set forth in SEQ ID NO: 1 or 2.

18. The method of claim 17, wherein the CD24 protein comprises a protein tag, wherein the protein tag is fused at the N-terminus or C-terminus of the CD24 protein.

19. The method of claim 18, wherein the protein tag comprises a Fc region of a mammalian immunoglobulin (Ig) protein.

20. The method of claim 19, wherein the Ig protein is a human Ig protein.

21. The method of claim 20, wherein the Fc region comprises a hinge region and CH2 and CH3 domains of IgG1, IgG2, IgG3, IgG4, or IgA.

22. The method of claim 20, wherein the Fc region comprises a hinge region and CH2, CH3 and CH4 domains of IgM.

23. The method of claim 21, wherein the CD24 protein comprises the sequence set forth in SEQ ID NO: 6, 11, or 12.

24. The method of claim 23, wherein the amino acid sequence of the CD24 protein consists of the sequence set forth in SEQ ID NO: 6, 11, or 12.

25. The method of claim 1, wherein the CD24 protein is soluble.

26. The method of claim 1, wherein the CD24 protein is glycosylated.


SEQUENCE LISTING (12 sequences)

SEQ ID NO: 1 - protein, 31 aa, Homo sapiens
  VARIANT (31)..(31): Valine or Alanine
SETTTGTSSNSSQSTSNSGLAPNPTNATTKX

SEQ ID NO: 2 - protein, 30 aa, Homo sapiens
SETTTGTSSNSSQSTSNSGLAPNPTNATTK

SEQ ID NO: 3 - protein, 27 aa, Mus musculus
NQTSVAPFPGNQNISASPNPTNATTRG

SEQ ID NO: 4 - protein, 26 aa, Homo sapiens
MGRAMVARLGLGLLLLALLLPTQIYS

SEQ ID NO: 5 - protein, 287 aa, Artificial
  Fusion protein
MGRAMVARLGLGLLLLALLLPTQIYSSETTTGTSSNSSQSTSNSGLAPNPTNATTKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 261 aa, Artificial
  Fusion protein
SETTTGTSSNSSQSTSNSGLAPNPTNATTKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 288 aa, Artificial
  Fusion protein
MGRAMVARLGLGLLLLALLLPTQIYSSETTTGTSSNSSQSTSNSGLAPNPTNATTKVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 288 aa, Artificial
  Fusion protein
MGRAMVARLGLGLLLLALLLPTQIYSSETTTGTSSNSSQSTSNSGLAPNPTNATTKAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 29 aa, Macaca fascicularis
TVTTSAPLSSNSPQNTSTTPNPANTTTKA

SEQ ID NO: 11 - protein, 262 aa, Artificial
  Fusion protein
SETTTGTSSNSSQSTSNSGLAPNPTNATTKVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 262 aa, Artificial
  Fusion protein
SETTTGTSSNSSQSTSNSGLAPNPTNATTKAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK